(19) United States
(12) Patent Application Publication
(10) Pub. No.: US 2003/0129194 A1
(43) Pub. Date: Jul. 10, 2003

(54) Targeted therapeutic delivery of vitamin D compounds

(75) Inventors: Richard B. Mazess, Madison, WI (US); Charles W. Bishop, Madison, WI (US)

Correspondence Address: MICHAEL BEST & FRIEDRICH, LLP, ONE SOUTH PINCKNEY STREET, P O BOX 1806, MADISON, WI, 53701

(73) Assignee: Bone Care International, Inc., Middleton, WI

(21) Appl. No.: 10/251,905

(22) Filed: Sep. 20, 2002

Related U.S. Application Data
(63) Continuation-in-part of application No. 09/402,636, filed on Apr. 26, 2000.
Application No. 09/402,636 is a 371 of international application No. PCT/US98/02899, filed on Feb. 13, 1998.
(60) Provisional application No. 60/038,364, filed on Feb. 13, 1997.

(51) Int. Cl. A61K 39/395; A61K 31/727; A61K 31/66; A61K 31/59

(52) U.S. Cl. 424/178.1; 514/102; 514/167; 514/54; 514/56

(57) ABSTRACT

The present invention is directed to a conjugate which includes at least one vitamin D moiety thereof and at least one targeting molecule moiety to pharmaceutical compositions of the conjugate, and to methods for using the conjugate for target-specific delivery of vitamin D or analogs thereof to tissues in need thereof. When a particularly preferred form is administered to a patient, the targeting molecule component of the conjugate of this invention seeks out and binds to a tissue of interest, such as bone or tumor tissue, where the vitamin D has a therapeutic effect.

### CROSS-REFERENCE TO RELATED APPLICATIONS

This patent application is a continuation-in-part of U.S. application Ser. No. 09/402,636, filed Feb. 13, 1998, which is a 371(a) of international patent application no. PCT/US98/02899, filed Feb. 13, 1998, which claims priority, under 35 U.S.C. §119, to provisional patent application No. 60/038,364, filed Feb. 13, 1997.

### STATEMENT REGARDING FEDERALLY SPONSORED RESEARCH OR DEVELOPMENT

Not applicable.

### BACKGROUND OF THE INVENTION

This invention relates generally to the targeted therapeutic delivery of vitamin D compounds and, in particular, delivery to bone and tumor tissue.

Too often the practicing clinician is faced with an unresolvable dilemma. To effectively achieve therapy against a disease, it becomes necessary to balance the devastation wrought by the illness against noxious effects conferred by the drugs used to treat it. Although therapy should represent a complete tolerance to the dose regimen, in absolute terms, it frequently delineates a compromise position, thereby, effecting only an “acceptable” level of treatment.

In the absence of preventive measures to combat the onset of disease, the next ideal alternative is to design a drug that specifically recognizes the origin of the disorder and then corrects it. This so-called “magic bullet” or site-specific drug delivery concept is not a new concept. Such a concept is directed to linking the action of drugs to specific receptor-mediated events. These concepts still serve as a primary foundation for the continuing development of drugs and antibodies.

Chemical modification of a drug, in some cases enhances its target organ specificity. For example, brain-targeted delivery systems based on the dihydropyridine-pyridinium salt redox interconversion have been developed for compounds such as estradiol and ethinylestradiol. Brewster et al., 31 J. Med. Chem. 244 (1988). Covalent coupling of some sugars to drugs can enhance their uptake by the liver. Ponpinom et al., in Receptor Mediated Targeting of Drugs, (Gregoriadis et al, eds.) NATO ASI series, Plenum Press, New York, 1983, p. 53.

In most cases, however, a specific carrier is needed that is designed for transport and delivery of the drug to its target tissue. In such cases, the distribution characteristics of the drug itself are essentially irrelevant, since the carrier characteristics determine whether the drug is delivered to the target cells. However, once delivered to the target cells, it still will be necessary to consider the distribution of the drug at its intracellular site.

A number of particles have been proposed as drug carrier systems. Recent interest has focused on monoclonal antibodies and colloidal delivery systems such as liposomes and polymeric microspheres or microcapsules formed of biocompatible polymers. See, e.g., Davis et al. in Site-Specific Drug Delivery, (Tomlinson et al. eds.), John Wiley, New York, 1986, p.93; Roth et al., U.S. Pat. No. 5,879,713; Use of Hepatoptes (Charodhury, N. L., et al., J. Biol. Chem. 268, 11265 (1993)) and immunoliposomes has also been reported.

Soluble molecules have also been proposed as drug carriers, including oligonucleotides, lectins, poly-L-lysine, virosomes, insulin, dextrans, HCG, dipeptides and even cellular systems such as erythrocytes and fibroblasts. See, e.g., Poznansky et al., 36 Pharmacol. Rev. 277 (1984). Still others have suggested and attempted to use lipoproteins as drug carriers, especially low-density lipoproteins. See, e.g., Counsell et al., 25 J. Med. Chem. 1115 (1982). See, also, Takle, et al., U.S. Pat. No. 5,891,689; Chari et al., U.S. Pat. No. 5,846,545.

The need for site-specific drug delivery is perhaps highest in pathological conditions such as cancer and severe viral infections. Side effects of the drugs utilized in the treatment of neoplastic diseases often are quite severe. Other fast-growing tissues such a bone marrow and gastrointestinal border cells are also affected by the antineoplastic drug, and therapy often must be stopped.

The need for site-specific drug delivery is also high in bone conditions, particularly osteoporosis. With the recognition that osteoporosis occurs to some extent in all postmenopausal women, the site-specific delivery of bone agents to the mineralized bone matrix has been proposed. Certain agents are known for their bone-seeking characteristics or bone affinity, and the linkage of such bone-seeking agents to enzymes, steroids, or hormones to provide a bone-specific drug delivery agent has been advanced. For example, it has been proposed to join a bone-seeking agent, such as tetracycline, to a carbonic anhydrase inhibitor through a bridging agent to provide compounds for the treatment of or prophylaxis of degenerative bone diseases. See, European Patent Application No. 201,057.

Further, it has been taught to link a hormone, e.g., calcitonin or insulin-like growth factor, to an amino methylene bisphosphonic acid. See, Japanese Patent Application No. 2104-593A. U.S. Pat. No. 5,183,815 discloses that the linkage of a steroid to an alkyl bisphosphonic acid can exhibit a localized therapeutic effect on bone. European Patent Application No. 0 512 844 A1 discloses linkage of a bone growth factor, such as transforming growth factor-beta, to bone-targeting molecules such as tetracycline, calcitonin, bisphosphonates, polyaspartic acid, polyglutamic acid, aminophosphosugars, or estrogens to provide a local bone-augmenting formation agent.

Vitamin D has long been established as having an important biologic role in bone and mineral metabolism. It is well known that vitamin D plays a critical role in stimulating calcium absorption and regulating calcium metabolism. The discovery of active forms of vitamin D, (M. F. Holick et al., 68 Proc. Natl. Acad. Sci. USA, 803-804 (1971); G. Jones et al., 14 Biochemistry, 1250 (1975) and active vitamin D analogs (M. F. Holick et al., 180 Science, 190 (1973); H. Y. Lam et al., 186 Science, 1038 (1974)), caused much excitement and speculation about the usefulness of these vitamin D compounds in the treatment of bone depletive disorders.

Animal studies examining the effects of these active vitamin D compounds suggested that such agents would be useful in restoring calcium balance. Further, an early clinical study indicated that administration of 0.5 μg/day of 1α,25-dihydroxyvitamin D₃, the hormonally active form of vitamin D₃, to a group of postmenopausal women improved the intestinal calcium absorption as well as the calcium balance in the women. On this basis, U.S. Pat. No. 4,225,596 (“'596 patent”) described and claimed the use of 1α,25-dihydroxyvitamin D₃ for increasing calcium absorption and retention. Such use also was claimed in the same patent for compounds 1,25-dihydroxyvitamin D₂, and 1α-hydroxyvitamin D₂, which the patent teaches are “eminently suitable and readily substitutable for the 1,25 dihydroxycholecalciferol [1α,25-dihydroxyvitamin D₃].”

The best indicator of the efficacy of vitamin D compounds in the prevention or treatment of depletive bone disorders, however, is bone itself rather than calcium absorption or calcium balance. More recent clinical data indicates that, at the dosage ranges taught in the '596 patent, 1α,25-dihydroxy-vitamin D₃ has, at best, modest efficacy in preventing or restoring loss of bone mass or bone mineral content (S. M. Ott and C. H. Chesnut, 110 Ann. Int. Med. 267 (1989); J. C. Gallagher et al., 113 Ann. Int. Med. 649 (1990); J. Aloia et al., 84 Amer. J. Med. 401 (1988)).

These clinical studies with 1α,25-dihydroxyvitamin D₃, and another conducted with 1α-hydroxyvitamin D₃ (M. Shiraki et al., 32 Endocrinol. Japan 305 (1985)), indicate that the capacity of these two vitamin D compounds to restore lost bone mass or bone mineral content is dose-related. These studies also indicate, however, that at the dosage ranges required for either of the compounds to be truly effective, toxicity in the form of hypercalcemia and hypercalciuria becomes a major problem. Specifically, attempts to increase the amount of 1α,25-dihydroxy-vitamin D₃ above 0.5 μg/day have frequently resulted in toxicity. At dosage levels below 0.5 μg/day, no effects are observed on bone mass or mineral content. (See G. F. Jensen et al., 16 Clin. Endocrinol. 515 (1982); C. Christiansen et al., 11 Eur. J. Clin. Invest. 305 (1981)). Two μg/day of 1α-hydroxyvitamin D₃ was found to have efficacy in increasing bone mass in patients exhibiting senile osteoporosis (O. H. Sorensen et al., 7 Clin. Endocrinol. 169S (1977)). Data from clinical studies in Japan, a population that has low calcium intake, indicate that efficacy is found with 1α-hydroxyvitamin D₃ when administered at 1 μg/day (M. Shiraki et al., 32 Endocrinol. Japan 305 (1985); H. Orimo et al., 3 Bone and Mineral 47 (1987)). At 2 μg/day, however, toxicity with 1α-hydroxy-vitamin D₃ occurs in approximately 67 percent of the patients, and at 1 μg/day, this percentage is approximately 20 percent.

More recently, other roles for vitamin D have come to light. Specific nuclear receptors for 1α,25-dihydroxyvitamin D₃ have been found in cells from diverse organs not involved in calcium homeostasis. For example, Miller et al., 52 Cancer Res. 515 (1992), have demonstrated biologically active, specific receptors for 1α, 25-dihydroxyvitamin D₃ in the human prostatic carcinoma cell line, LNCaP.

More specifically, it has been reported that certain vitamin D compounds and analogs are potent inhibitors of malignant cell proliferation and inducers/stimulators of cell differentiation. For example, U.S. Pat. No. 4,391,802 issued to Suda et al. discloses that 1α-hydroxyvitamin D compounds, specifically 1α,25-dihydroxyvitamin D₃ and 1α-hydroxyvitamin D₃, possess potent antileukemic activity by virtue of inducing the differentiation of malignant cells (specifically leukemia cells) to nonmalignant macrophages (monocytes), and are useful in the treatment of leukemia. In another example, Skowronski et al., 136 Endocrinology 20 (1995), have reported antiproliferative and differentiating actions of 1α,25-dihydroxyvitamin D₃ and other vitamin D₃ analogs on prostate cancer cell lines.

Previous proliferation studies, such as those cited above, focused exclusively on vitamin D₃ compounds. Even though such compounds may indeed be highly effective in differentiating malignant cells in culture, their practical use in differentiation therapy as anticancer agents is severely limited because of their equally high potency as agents affecting calcium metabolism. At the levels required in vivo for effective use as antileukemic agents, these same compounds can induce markedly elevated and potentially dangerous blood calcium levels, by virtue of their inherent calcemic activity. That is, the clinical use of 1α,25-dihydroxyvitamin D₃ and other vitamin D₃ analogs as anticancer agents is precluded, or severely limited, by the risk of hypercalcemia. This indicates a need for compounds with greater specific activity and selectivity of action, i.e., vitamin D compounds with antiproliferative and differentiating effects but which have less calcemic activity than therapeutic amounts of the known compounds or analogs of vitamin D.

Virtually nothing in the art proposes materials or methods for the targeted delivery of vitamin D compounds to specific target tissue, e.g., bone or malignancy sites, such as prostatic cancer cells.

### BRIEF SUMMARY OF THE INVENTION

The present invention provides conjugates that include vitamin D compounds or analogs and a targeting molecule having an ability for site-specific delivery of the vitamin D. Specific conjugates include a bone-therapeutic conjugate and an anti-tumor or antihyperproliferative (or antineoplastic) conjugate. The present invention also provides pharmaceutical formulations of such a conjugate, and methods of site-specific delivery of a vitamin D moiety of the conjugate to a tissue of interest in a patient. The conjugates are such that the vitamin D moiety maintains its biological effectiveness. The present invention provides surprisingly useful means for delivery of vitamin D compounds, analogs or derivatives to tissues of interest (i.e., to target tissue).

The conjugates in accordance with the present invention comprise at least one vitamin D moiety associated with a targeting molecule moiety having an affinity for the target tissue. In one embodiment of the invention, the conjugate includes at least one vitamin D moiety associated with the targeting molecule moiety via at least one connecting group, such as a bond between the vitamin D moiety and the targeting molecule moiety, a bifunctional connector, or other connectors such as a biotin-avidin linkage. In another embodiment, the conjugate includes a targeting molecule moiety having an affinity for the tissue of interest, associated with at least one vitamin D moiety and with a second therapeutic agent other than vitamin D.

The pharmaceutical compositions of the present invention include a conjugate of at least one vitamin D moiety associated with at least one targeting molecule moiety, and a suitable pharmaceutically acceptable carrier.

In another embodiment, the present invention provides a method for site-specific delivery of a vitamin D compound or analog, a method which includes administering to a patient a therapeutically effective dose of a conjugate in a pharmaceutically acceptable carrier, wherein the conjugate has at least one vitamin D moiety associated with at least one targeting molecule via a connector, and wherein the targeting molecule has an affinity for a tissue of interest, i.e., a target tissue. The method is designed to effect site-specific delivery of the vitamin D moiety to the tissue of interest in the patient.

In all embodiments of the present invention, target tissue of particular interest includes bone, tumor, and skin.

The conjugates, pharmaceutical compositions, and method of the present invention allow for target-specific delivery of vitamin D to a specific tissue in a patient. By specifically targeting and delivering a vitamin D compound as part of the conjugates of this invention, one can effect delivery of the compound to the targeted tissue by administering relatively small amounts of the conjugate to a patient compared to dosage amounts of the compound that are required when administering the compound itself by conventional means. By reducing the amount of vitamin D compound administered, the present invention significantly reduces the risk of hypercalcemia and other side-effects of administration of vitamin D compounds or analogs to patients compared to using conventional means of administration.

Other advantages and a fuller appreciation of specific adaptations, compositional variations, and physical attributes will be gained upon an examination of the following detailed description of preferred embodiments, taken in conjunction with the figures of the drawing. It is expressly understood that the drawings herein are for the purpose of illustration and description only, and are not intended as a definition of the limits of the invention.

### BRIEF DESCRIPTION OF THE DRAWINGS

The preferred exemplary embodiment of the present invention will hereinafter be described in conjunction with the appended drawings, wherein like designations refer to like elements throughout, and in which:

FIG. 1 illustrates a reaction scheme for the preparation of a conjugate of 1α,24-dihydroxyvitamin D₂ (“1α,24-(OH)₂D₂”) and aminoalkyl-1,1-bisphosphonate linked at C-24 of the vitamin D moiety;

FIGS. 2A and 2B illustrate a reaction scheme for the preparation of a conjugate of 1α,24-(OH)₂D₂ and aminoalkyl-1,1-bisphosphonate linked at C-1 of the vitamin D moiety;

FIG. 3 illustrates a reaction scheme for the preparation of a conjugate of 1α,24-(OH)₂D₂ and aminoalkyl-1,1-bisphosphonate linked at C-3 of the vitamin D moiety;

FIGS. 4A and 4B illustrate a reaction scheme for the preparation of a conjugate of 1α,25-dihydroxyvitamin D₃ (1α,25-(OH)₂D₃) and aminoalkyl-1,1-bisphosphonate linked at C-1 of the vitamin D moiety;

FIG. 5 illustrates a reaction scheme for the preparation of a conjugate of 1α,25-(OH)₂D₃ and aminoalkyl-1,1-bisphosphonate linked at C-3 of the vitamin D moiety;

FIG. 6 illustrates a reaction scheme for the preparation of a conjugate of 1α, 25-(OH)₂D₃ and aminoalkyl-1,1-bisphosphonate linked at C-25 of the vitamin D moiety; and

FIG. 7 is a schematic diagram for preparation of a conjugate of vitamin D and a monoclonal antibody, utilizing a biotin-avidin connector.

### DETAILED DESCRIPTION OF THE INVENTION

The present invention relates to the use of vitamin D conjugates in targeting applications. The present invention is especially useful in site-specific delivery of a vitamin D compound or analogs to bone and tumor cells. Accordingly, the present invention will now be described in detail with respect to such endeavors. However, those skilled in the art will appreciate that such a description of the invention is meant to be exemplary only and should not be viewed as limiting the full scope thereof.

The present invention is characterized by an ability for site-specific targeting of vitamin D compounds using conjugates of a vitamin D compound and a targeting molecule having affinity for a tissue of interest. For example, a conjugate of a vitamin D compound and a bone-affinity agent is designed to transport and deliver the vitamin D compound to bone. These attributes are achieved through a novel combination of physical and chemical features of the conjugates in accordance with the present invention.

In the following description of the invention, process steps are carried out at room temperature and atmospheric pressure, unless otherwise specified.

As used herein, the term “targeting molecule” or “target molecule” refers to a molecule that binds to or influences metabolism of the tissue of interest i.e., target tissue. For example, bone-targeting agents may include bone-seeking molecules such as tetracycline, calcitonin, bisphosphonates, chelators, phosphates, polyaspartic acid, polyglutamic acid, aminophosphosugars, estrogen, peptides known to be associated with mineral phase of bone such as osteonectin, bone sialoprotein and osteopontin, protein with bone mineral binding domains, and the like. Targeting molecules may also include peptides of a repetitive acidic amino acid which may work as a carrier for vitamin D compounds. Examples of suitable small acidic peptides include, but are not limited to, (Asp)₆ or (Glu)₆.

Bone-targeting molecules may also include molecules which themselves affect bone resorption and bone formation rates, such as bisphosphonates, estrogens and other steroids, such as dehydroepiandrosterone (DHEA). These bone-seeking molecules may also possess bone growth therapeutic properties and/or result in a synergistic or additive effect with the vitamin D compound on bone resorption or formation.

Skin-seeking molecules include certain metal ion-amino acid chelates; prostate-seeking molecules include certain steroids such as DHEA. Liver-seeking molecules include triglycerides, particularly medium-chain triglycerides. Tumor-seeking agents include certain antibodies, such as antibodies for vascular permeability factor and monoclonal antibodies, oxidized glycosylated proteins, polylysine, human serum albumin, dextrans, peptides and proteins that have an affinity for particular receptors, such as gastrin releasing peptide receptor, epidermal growth factor receptor, platelet-derived growth factor receptor, tumor necrosis factor receptor, fibroblast growth factor receptor, insulin-like growth factor receptor, transfertin receptor, laminin receptor, cytokine receptors, fibronectin receptor, interleukin receptor, interferon receptors, bombesen/gastrin-releasing peptide receptor, somatostation receptor, etc., polyanionic compounds and polymers, such as sumarin and analogues and derivatives of sumarin, polysulphated compounds and polymers, such as heparin, heparan sulfate, chrondroitin sulfate, keratan sulfate, dermatan sulfate, sulfated chitin, sulfated chitosan, sulfated alginic acid, pentosan polysulfate, sulfated cyclodextrins, and synthetic organic polymers including polystyrene sulfonate, sulfated polyvinyl alcohol, polyvinyl sulfate, and polyethylene sulfonate, and analogs of peptide hormones, such as LH-RH, dombesin, and somatostatin.

As used herein, the term “vitamin D molecule” or “vitamin D moiety” or “vitamin D molecule moiety” refers to an “activated vitamin D” or “active vitamin D” which is intended to include any biologically active vitamin D compound. It is known that vitamin D compounds display a variety of biological activities, e.g., in calcium and phosphate metabolism (see, e.g., U.S. Pat. No. 5,104,864), as an antineoplastic agent (see, e.g., U.S. Pat. No. 5,763,429), and as an anti-hyperparthyroid agent (see, e.g., U.S. Pat. No. 5,602,116), and it is contemplated that any of the biologically active forms of vitamin D can be used in the formulations in accordance with the present invention. Generally, an active vitamin D compound or analog is hydroxylated in at least the C-1, C-24 or C-25 position of the molecule.

Examples of vitamin D compounds suitable for formulations of the present invention include, without limitation, 1 α-dihydroxyvitamin D₂ (doxercalciferol), 1α,2-dihydroxyvitamin D₄, 1α,24-dihydroxyvitamin D₂, 1α,25-dihydroxyvitamin D₃ (calcitriol), 1α hydroxyvitamin D₃ (α-calcidol) 1α,25-dihydroxyvitamin D₂, 1α,25-dihydroxyvitamin D₄, and 1α,24,25-dihydroxyvitamin D₂, seocalcitol (EB-1089), calcipotriol, 22-oxacalcitriol (maxacalcitol), fluorinated compounds such as falecalcitriol, and 19-nor compounds such as paricalcitol. Among those compounds having a chiral center, e.g., in the sidechain, such as at C-24, it is understood that both epimers (e.g., R and S) and the epimeric mixture are within the scope of the present invention.

As used herein, the terms “tissue of interest” or “target tissue” are meant to refer to a desired target or site in a living organism, e.g., a human body, for treatment or for placement of a vitamin D compound or analog. A targeting molecule may bind to or have affinity for a specific component of a target tissue. For example, several of the peptide targeting molecules, discussed above, have an affinity for and tend to bind to hydroxyapatite found in a living organism's bone or teeth. Hydroxyapatite (HA), the chemical formula for which is Ca₁₀(PO₄)₆(OH)₂, is a major inorganic component and constituent in the matrices of hard tissues such as bone and teeth, but does not exist in soft tissues.

The term “treat” or “treatment” is meant to refer to repair, prevention, alleviation, amelioration, prophylaxis of a diseased or defective tissue of interest (e.g., prevention of loss of bone) as well as inhibition of abnormal growth, such as hyperproliferation of cells, and promotion of cell differentiation.

As used herein, the term “therapeutic agent” refers to a material or substance which has or exhibits healing powers when administered to or delivered to the tissue of interest. The term “bone-therapeutic agent” is used herein to refer to a specific type of therapeutic agent, one which treats bone diseases or disorders when delivered or administered to bone. Such bone-therapeutic agents may, e.g., prevent or stabilize bone loss, or promote bone growth. Examples of bone-therapeutic agents include vitamin D compounds, conjugated estrogens or their equivalents, antiestrogens, calcitonin, bisphosphonates, calcium supplements, cobalamin, pertussis toxin, boron and other bone growth factors such as transforming growth factor beta, activin or bone morphogenic protein.

As used herein, the terms “associated with” or “association” or “bound to” are meant to refer to attachment or linkage of one component of the conjugate (e.g., the vitamin D moiety) to another, or, e.g., a vitamin D moiety and connector to another component of the conjugate (e.g., the targeting molecule), via covalent bonding, hydrogen bonding, metallic bonding, van der Wall forces, ionic bonding, coulombic forces, hydrophobic or hydrophilic forces, adsorption or absorption, chelate type associations, or any combination(s) thereof. Also contemplated within the meaning of “associated with” or “association” or “bound to” are solution or dispersion forces wherein the vitamin D moiety may be dissolved and thus solvated with a solvent.

The terms “moiety” and “component” used in connection with vitamin D or in connection with a targeting molecule (or in connection with a therapeutic agent) are meant to refer to vitamin D or the targeting molecule in the conjugated forms disclosed herein, i.e., after association occurs. For example, association between the vitamin D analog and the targeting molecule may occur at any position on the vitamin D analog molecule depending on the functionality of the targeting molecule. For example, a bisphosphonate-or an amide may suitably link at positions on a vitamin D compound or a vitamin D analog molecule having a hydroxyl group, such as at positions C-1, C-3, C-24, or C-25.

The term “lower” in reference to chemical functional groups refers to C₁-C₄, straight or branched chains.

It also is understood that any numerical value recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between the lowest value and the highest value enumerated are to be considered to be expressly stated in this application.

The conjugates in accordance with the present invention include at least one vitamin D moiety (herein designated as “D”) associated with at least one targeting molecule or moiety (herein designated as “T”) and include those represented by formula (I):

(D)ₘ*(T)ₙ (I)

wherein n and m represent integers of 1 or greater; and * indicates that the targeting moiety (T) is associated with the vitamin D compound, analog, component or moiety (D). It is understood that as used herein, the term “vitamin D” includes all compounds having the conventional vitamin D structure of A, C and D rings and C-17 side chain as well as previtamin D compounds which are the thermal isomers of their corresponding vitamin D forms, in which the basic structures may be substituted, unsubstituted or modified, e.g., 19-nor compounds. It is also understood that the vitamin D moiety maintains its biological effectiveness in the conjugate, e.g., its beneficial effect with respect to bone, or in the case of a previtamin D, isomerizes to its corresponding D form having the biological effectiveness.

Vitamin D compounds and analogs operable in the present invention are suitably represented by formula (II):

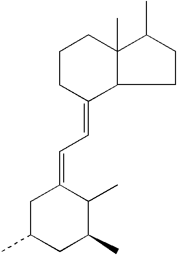

wherein R¹ is H or OH; Z represents a saturated or unsaturated, substituted or unsubstituted, straight-chain or branched C₁-C₁₈ hydrocarbon group; Y is a ═CH₂ group; and t is 0 or 1, such that when t is 0, the compound of formula (II) is a 19-nor compound. Z is suitably a side chain represented by formula (IIIA):

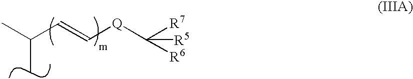

wherein m is 0 or 1; R⁵ is H or OH; R⁶ and R⁷ are independently H, OH, lower alkyl, lower fluoroalkyl, O-lower alkyl, O-lower acyl, O-aromatic alkyl, lower cycloalkyl or, taken together with the carbon to which they are bonded (i.e., C-25), form a C₃-C₈ cyclohydrocarbon ring; and Q is —C═C—, —C≡C—, or

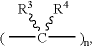

wherein n is 0 or an integer from 1 to 7, R³ is CH₃ or H, and R⁴ is H or OH. For example, Z includes a cholesterol or ergosterol side chain represented by formula (IIIB):

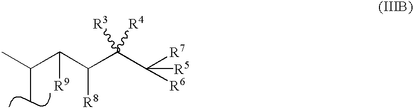

wherein R⁸ and R⁹ are each H or taken together form a double bond between C-22 and C-23, R³ is CH₃ or H; R⁴ and R⁵ are independently H or OH; and R⁶ and R⁷ are independently H, OH, lower alkyl, lower fluoroalkyl, O-lower alkyl, O-lower acyl, O-aromatic acyl, lower cycloalkyl or taken together with the carbon to which they are bonded (i.e., C-25) form a C₃-C₈ cyclocarbon ring.

Also included as vitamin D compounds within the scope of the present invention are previtamin D compounds, for example, 1α-hydroxyprevitamin D which may include the common cholesterol and ergosterol side chains that may optionally be substituted, for example, hydroxysubstituted, e.g., at C-24 or C-25. Previtamin D compounds are the thermal isomers of the corresponding vitamin D compounds, e.g., 1α-hydroxyprevitamin D₃ is the thermal isomer of 1α-hydroxyvitamin D₃, and exists in thermal equilibrium with same.

The targeting molecules are suitably those which, when used as a component of the conjugate of formula (I), result in at least a portion of the conjugate, specifically the vitamin D component of the conjugate, being delivered to a desired target (e.g., a targeted cell, a targeted organ, a targeted component of a tissue of interest, a tumor, etc.). The targeting molecules suitably include chemical functionalities exhibiting target specificity, e.g., hormones (e.g., biological response modifiers), and antibodies (e.g., monoclonal or polyclonal antibodies), or antibody fragments having the requisite target specificity, e.g., to specific cell-surface antigens. Included among targeting molecules exhibiting specific affinity for bone are bisphosphonates, small acidic peptides, tetracycline, polymalonates and dehydroepiandrosterone.

The conjugates of formula (I) are suitably prepared under conditions (such as particular pH, temperature or salt concentrations) which are not detrimental to the particular conjugate components and in the presence of a suitable solvent when required. For example, to control pH, a buffer or addition of a suitable acid or base is used. The reaction conditions are dependent on the type of association (*) to be formed between the vitamin D compound or analog (D) and the targeting molecule (T) in order to produce the conjugate.

Suitably, the molar ratio of T:D in the conjugates of formula (I) is 1:1.

Also within the scope of the present invention are those conjugates in which D is associated with T via connector (herein designated as “G”) between D and T; these conjugates are represented by the formula (IV):

[(T)ₙ-(G′)_{f}]_{g}*[(G″)ₕ-(D)ₘ]ₖ (IV)

wherein each G′ represents the same or different connecting group; each G″ represents the same or different connecting group; g and k each individually represent an integer of 1 or greater; f and h each individually represent an integer of 0 or greater; — indicates a bond in instances where a connecting group is present; n and m are as previously defined herein; and * indicates that the targeting molecule is associated with the vitamin D component via connector G′ or G″ or via both connectors, in instances when both are present. It is understood that D maintains its biological effectiveness in the conjugate or, when D is a previtamin D, isomers to its corresponding vitamin D form having biological effectiveness. It is also understood that T maintains its targeting affinity in the conjugate.

In those cases wherein f or h is zero (and g and k are 1), the conjugate of formula (IV) is simply represented by formula (V):

Dₘ-G-Tₙ (V)

In such cases, G is suitably a bifunctional connector, e.g., polyglutamic acid or polyaspartic acid, or a linkage group formed by modification of D and/or T and with subsequent bond formation.

Suitable connecting groups for use in forming the conjugates of formula (IV) are those which link the vitamin D moiety to the targeting molecule moiety without significantly impairing the biological effectiveness of the vitamin D, and without significantly impairing the affinity of the targeting molecule component of the conjugate to the target tissue. The connectors form a link between the targeting moiety and the vitamin D moiety of the conjugate, a link which must be of sufficient stability to remain intact, at least until the conjugate is delivered to the region proximate the target. Under some circumstances, the vitamin D delivered to the target region as part of the conjugate is more effective after at least one connector linking the targeting moiety and the vitamin D moiety is cleaved. In such cases, at least one such connector is preferably cleaved once the conjugate is delivered proximate the target. In other words, by cleaving the connectors in such cases, one can avoid any steric hindrance between the vitamin D and the targeting molecule which may exist in some of the conjugates of the present invention.

In one illustrated embodiment, a conjugate in accordance with the present invention includes an agent having an affinity for bone effective in treating bone disorders, i.e., the conjugate includes a vitamin D moiety and a bone-seeking agent. The bone-seeking agent may also be a bone-therapeutic agent, i.e., an agent. One such class of agents having bone affinity and bone therapeutic properties is bisphosphonates (“BP”) (also referred to as bisphosphonates or diphosphonates).

Examples of bisphosphonates include, but are not limited to 1-hydroxyethylidene-1,1-bisphosphonic ligand (etidronate), dichloromethylene bisphosphonic acid ligand, 3-amino-1-hydroxypropylidene-1-bisphosphonic acid ligand (pamidronate), alendronate, clodronate, ibandronate, rosedronate, tiludronate, zoledronate, and combinations thereof. For example, a specific bisphosphonate moiety which is suitably operable in the present invention is represented by formula (VI):

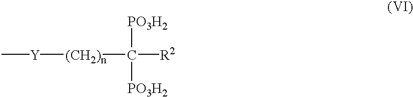

wherein R2 is H or OH, Y is NH, O or NR⁸ wherein R⁸ is H or C₁-C₄ alkyl, and n is an integer from 1 to 4. A bone-therapeutic conjugate in accordance with the present invention, wherein T is a BP, is represented by formula (VII):

D-BP (VII)

wherein BP is suitably linked at, e.g., the C-1, C-3, C-17, C-24, or C-25 position of the D moiety.

Specific bone-therapeutic conjugates suitable for use in the present invention, wherein T is a bisphosphonate, include conjugates of formula (VIII):

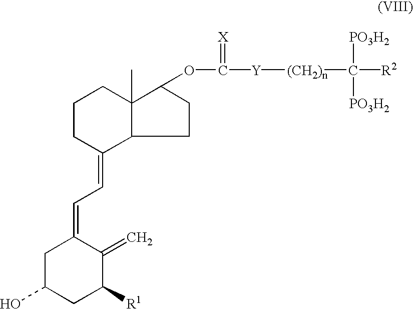

wherein R¹ is H or OH; R² is H or OH; X is O or S; Y is NH, O or NR wherein R is H or C₁-C₄ alkyl; n is 1-4; and pharmaceutically acceptable salts thereof, i.e., the bisphosphonate is linked to the vitamin D moiety at C-17.

Also provided are conjugates of formula (IX):

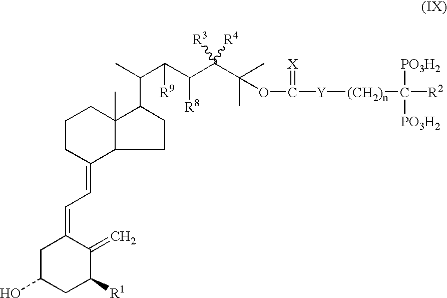

wherein R¹ is H or OH; R² is H or OH; R³ is CH₃ or H; R⁴ is H or OH, X is O or S; Y is NH, O or NR wherein R is H or C₁-C₄ alkyl; n is an integer from 1 to 4; R⁸ and R⁹ are each H or taken together form a double bond between C-22 and C-23; and pharmaceutically acceptable salts thereof, i.e., the bisphosphonate is linked at the C-25 position of the vitamin D moiety.

Also provided are conjugates of formula (X):

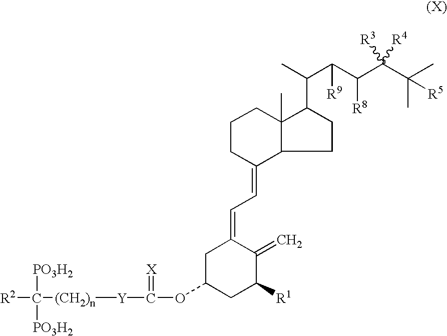

wherein R¹ is H or OH; R² is H or OH; R³ is CH₃ or H; R⁴ is H or OH, X is O or S; Y is NH, O or NR wherein R is H or C₁-C₄ alkyl; R⁵ is H or OH; n is an integer from 1 to 4; R⁸ and R⁹ are each H or taken together form a double bond between C-22 and C-23; and pharmaceutically acceptable salts thereof, i.e., the bisphosphonate is linked to the vitamin D moiety at C-3.

Also provided are conjugates of formula (XI):

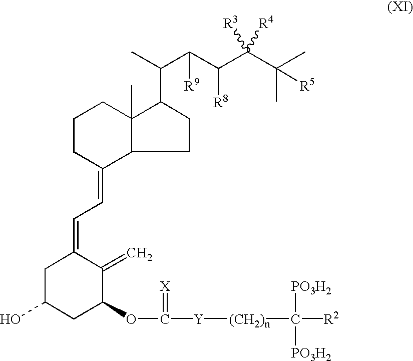

wherein R² is H or OH; R³ is CH₃ or H; R⁴ is H or OH, X is O or S; Y is NH, O or NR wherein R is H or C₁-C₄ alkyl; R⁵ is H or OH, n is an integer from 1 to 4, R⁸ and R⁹ are each H or taken together form a double bond between C-22 and C-23; and pharmaceutically acceptable salts thereof, i.e., the bisphosphonate linkage is at C-1 of the vitamin D moiety.

It is noted that typically the linkage between the bisphosphonate moiety and the vitamin D moiety is suitably through a hydroxyl on the vitamin D where the hydroxyl is converted to a

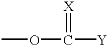

group and is linked to the amine or hydroxy group, i.e., Y, of the bisphosphonate to form a carbamate-type or carbonate-type linkage. X may be O or S. For example, a hydroxyl group may be contained in the vitamin D structure at C-1, C-3, C-24, C-25, and conjugation can be effected at any hydroxyl position but is suitably one of the above.

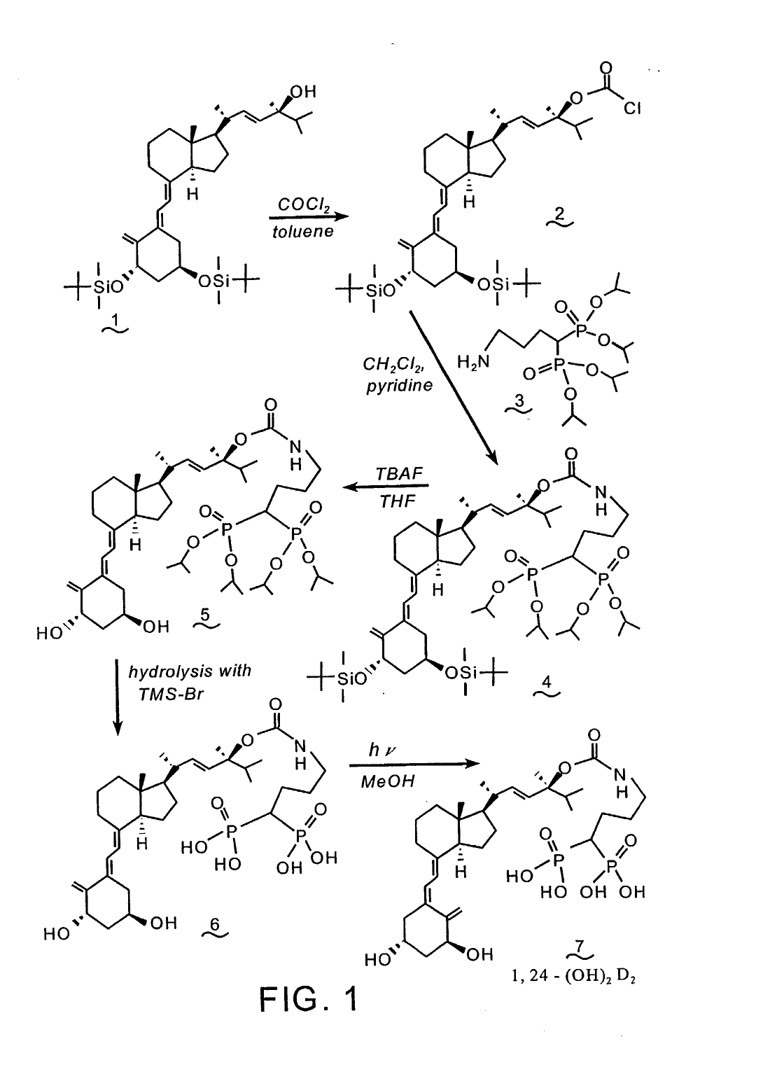

Synthesis of the conjugates of formula (I) (or (VII)), wherein T is a bisphosphonate, is suitably accomplished according to the schema presented in FIGS. 1-6. In general terms, the synthesis includes conversion of a hydroxyl of the vitamin D to a haloformate (e.g., a chloroformate) or thioformate group with subsequent reaction with the appropriate amino or hydroxyl group of the bisphosphonate to form a carbamate, thiocarbamate, carbonate or thiocarbonate linkage. If R² is hydroxy or the vitamin D compound contains one or more hydroxyl groups in addition to the desired hydroxyl group, these can be protected by conventional hydroxy-protecting groups, such as benzyl, silyloxy, etc., prior to the reaction that converts the desired hydroxyl to a haloformate or thioformate group.

Specifically, in the illustrated schema, the starting vitamin D compound or analog (when appropriate, hydroxyl protected) is reacted with phosgene to form a chloroformate, the chloroformate is reacted with an aminobutyl-1,1-bisphosphonate to form a carbamate linkage. Any protected hydroxyls are then deprotected.

FIG. 1 is an illustrative scheme for the synthesis of a conjugate of 1α,24-(OH)₂D₂ and aminoalkyl-1,1-bisphosphonate. As seen in FIG. 1, the 1α,24-(OH)₂D₂ starting material is protected by silyloxyl groups in the C-1 and C-3 positions. The protected D compound (1) is reacted with phosgene in toluene to form the chloroformate (2). The chloroformate (2) is reacted with tetraisopropyl 4-aminobutyl-1,1-bisphosphonate (i.e., hydroxyl-protected) in dichloromethane, and the reaction mix is purified via flash chromatography to yield the protected conjugate (4). The protected conjugate (4) is reacted with tetrahydrofuran (THF) and tetrabutylammonium fluoride (TBAF) to deprotect the C-1 and C-3 hydroxyls to yield the tetraisopropyl ester of the conjugate (5). The tetraisopropyl ester (5) is hydrolyzed with trimethyl silylbromide to form the conjugate bisphosphonic acid structure (6). Compound (6) and 9-acetylanthracene in methanol are irradiated, filtered, concentrated and lyophilized to yield the conjugate (7).

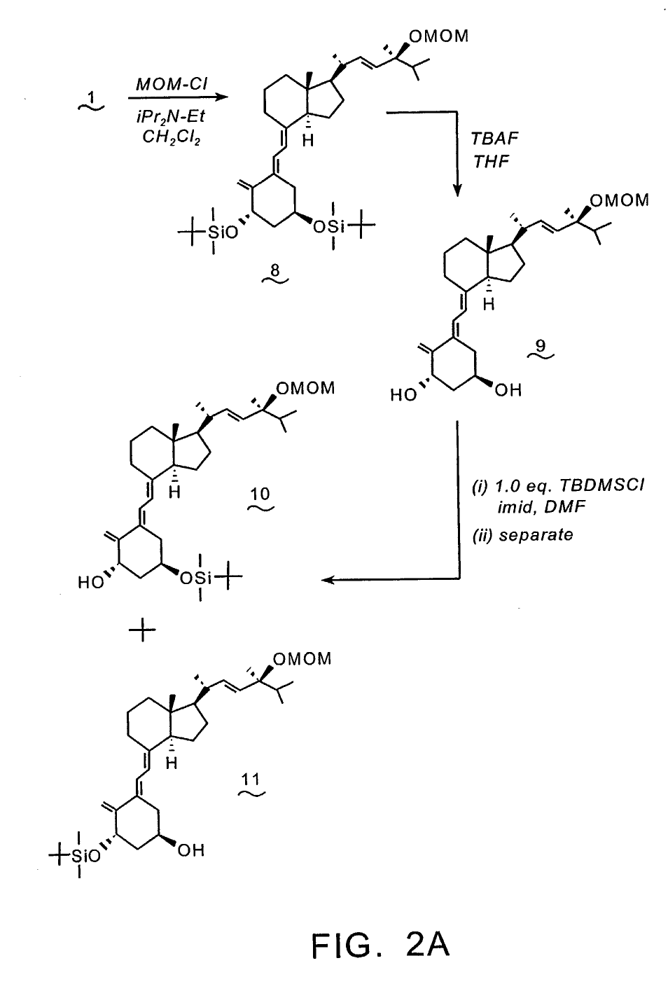

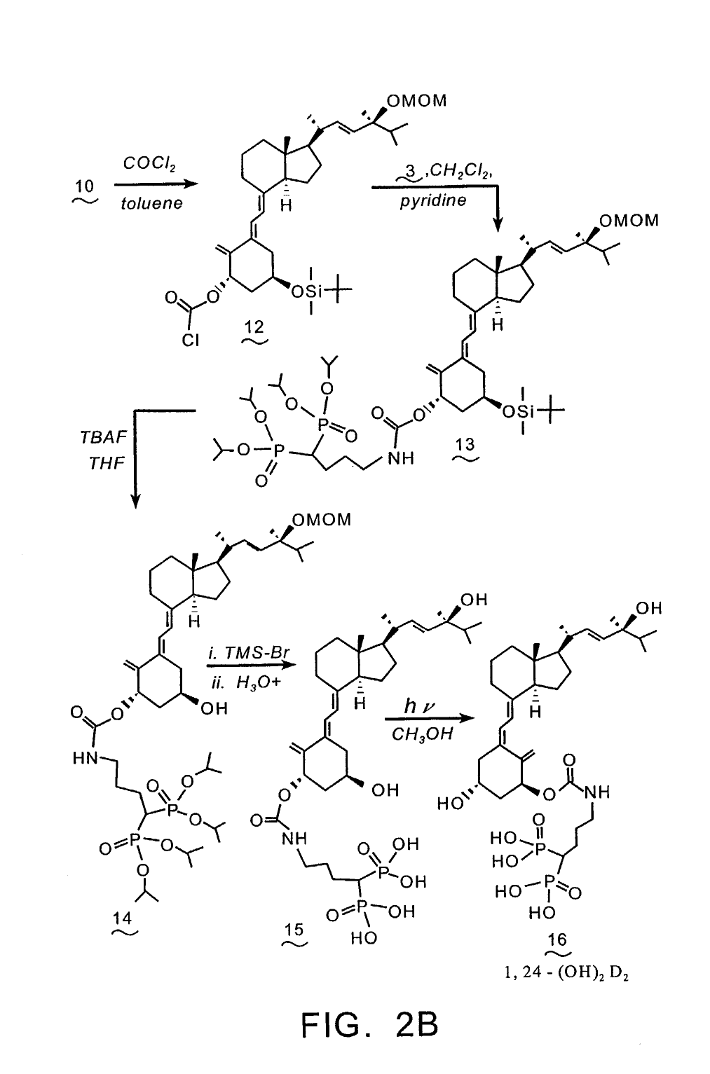

The syntheses illustrated in FIGS. 2-6 are discussed in detail in the Examples section, below.

The present invention also provides for targeted delivery of vitamin D which is conjugated with a related family of bisphosphonates, namely polyaminomethylenephosphonic acid ligands in a way that allows its preferential localization to skeletal tissue. Conjugates of vitamin D with polyaminomethylenephosphonic acid ligands are represented by formula (XII):

D-[(J)_{z}-L-AP] (XII)

wherein D is a vitamin D moiety; J is an acid cleavable linker which is covalently bonded to D; z is 0, 1 or 2; L is a linking moiety; and AP is a phosphonic acid ligand, or a polyaminomethylenephosphonic acid ligand.

Formulations for administering the compounds of formula (XII) to mammals, and methods for the use of the compounds of formula (XII) for targeted delivery to bone, and processes for preparing the compounds of formula (XII) are also contemplated by this invention.

The polyaminomethylenephosphonic acid ligands (AP of formula (XII)) are either covalently bonded to the D of formula (XII) (z=0), or have a cleavable linker (J) present (z=1). The AP ligands may be straight or branched-chain moieties, cyclic moieties, polymers, or aryl moieties, which ligands contain at least two, preferably three or more, nitrogen atoms. Suitable ligands include polyaminomethylenephosphonic acid ligands of one the following formulas:

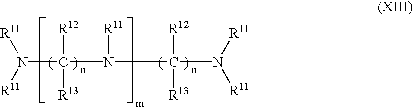

wherein each R¹¹ independently is hydrogen, C₁-C₄ alkyl, phenyl, hydroxy C₁-C₄ alkyl, —CH₂COOH, or —CH₂PO₃H₂ or an L moiety; with the proviso that only one of R¹ may be an L moiety and one L moiety must be present and with the proviso that at least one-half of the total R¹'s are —CH₂PO₃H₂; each R¹² and R¹³ independently is hydrogen, C₁-C₄ alkyl or L moiety; with the proviso that only one L moiety is present in formula (XIII); n is independently 2, 3 or 4; n″ is independently 2, 3 or 4; and m is 0 to 10; or

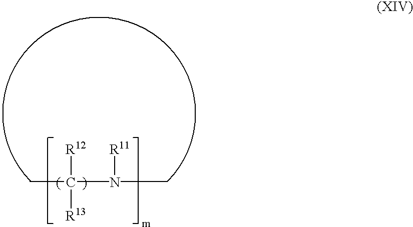

wherein R¹¹, R¹², R¹³, n and m are defined as before; or

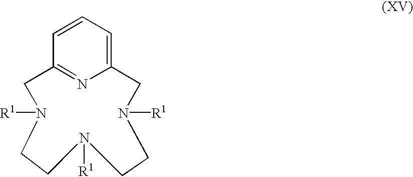

wherein R¹¹ is defined as before.

The linking moiety (L in formula (XII)) is represented by the formula:

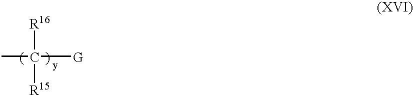

wherein G is hydrogen, NH² or

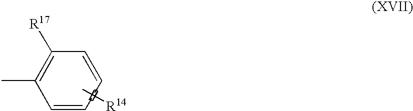

wherein R¹⁴ is an electrophilic group capable of being attached to protein; R¹⁵ and R¹⁶ are independently hydrogen or —COOH; with the proviso that when G is hydrogen, then one of R¹⁵ or R¹⁶ is COOH; R¹³ is hydrogen, hydroxy or C, -C₄ alkoxy; and y is 0, 1, 2, 3 or 4; with the proviso that when y is 1, 2, 3 or 4, the only one of R¹⁵ or R16 may be COOH.

For polyaminomethylenephosphonic acid ligands of the present invention, the following terms apply. The term “straight or branched-chain moieties” refers to an alkyl group having from 1 to about 100 carbon atoms which may be either a straight-chain moiety such as, for example, ethyl, propyl, n-butyl, n-dodecane and the like, or a branched-chain moiety such as, for example, isopropyl, tert-butyl, 2,5,7-trimethyldodecyl and the like. Both the straight and branched-chain moieties must contain at least 2 nitrogen atoms, preferably from 3 to 50 nitrogen atoms, and more preferably from 3 to 25. Some examples of these moieties include:

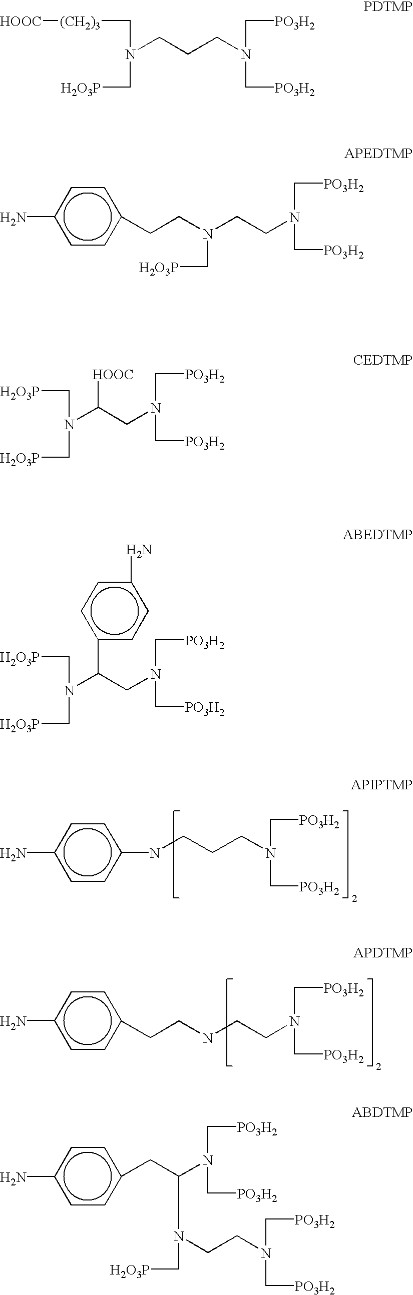

wherein PDTMP=(N-propylcarboxyl)ethylenediamine-N,N′,N′-trimethylene phosphonic acid; APEDTMP=[N-(4-aminophenyl)ethyl]ethylenediamine-N,N′,N′-trimethylene phosphonic acid; CEDTMP=1-(carboxyl)ethylenediamine-N,N,N′,N′-tetramethylene phosphonic acid; ABEDTMP=[1-(4-aminobenzyl)]ethylenediamine-N,N,N′,N′-tetramethylene phosphonic acid; APIPTMP=N-(4-aminophenyl)-N,N-bis-[propyl(iminodimethylenephosphonic acid)]; APDTMP=N-[(4-aminophenyl)ethyl]-N,N-bis-[ethyl(iminodimethylene phosphonic acid)]; and ABDTMP=N-[1-(4-aminobenzyl)-N,N′-ethylenediamine-N′,N′-ethylenediamine-N,N,N′,N″-pentamethylenephosphonic acid. ABEDTMP and ABDTMP are particularly suitable.

Small-peptide conjugation also provides a method of delivering a vitamin D moiety to a bone tissue. Hydroxyapatite (HA), a major inorganic component and constituent in the matrix of hard tissues such as bone and teeth, may act as a specific site in targeting bone tissue, to which a peptide conjugated vitamin D moiety may show affinity. In other words, conjugating a peptide with a vitamin D moiety may increase the affinity of a conjugated vitamin D moiety to hydroxyapatite (HA) component in a living organism's bone or teeth. As a result, peptides of repetitive acidic amino acid may be of value as carriers for vitamin D compounds to affected tissues of interest.

For example, several bone noncollagenous proteins having repeating sequences of acidic amino acids (Asp or Glu) in their structures have an affinity for and tend to bind to hydroxyapatite (HA). Osteopontin and bone sialoprotein, two major noncollagenous proteins in bone, have an Asp and Glu repeating sequence, respectively. Both osteopontin and bone sialoprotein have a strong affinity for and rapidly bind to HA. Therefore, conjugating vitamin D moieties with peptides associated with these and other noncollagenous proteins may be effective in targeting therapeutic delivery of the Vitamin D compounds to the bone or teeth because of the associated peptides' affinity to HA. (Asp)₆ conjugation may be a particularly effective delivery means because of the high affinity of (Asp)₆ to hydroxyapatite (HA), however (Glu)₆ may be just as effective.

In contrast to bisphosphonate conjugation, acidic peptides used in peptide conjugation tend to degrade in the resorption process, and may show no pharmacological effect. With bisphosphonate conjugation, the treated tissue tends to exhibit some biphosphonate effect. Comments and descriptions relating to formula (I) conjugates apply to small acidic peptide conjugates.

Also, preferred among the conjugates of formula (I) are those in which T is DHEA. The common steriodal structure of DHEA has a 17-keto group and a 1-hydroxy group. Either of these groups may suitably be linked to hydroxylated positions on the vitamin D, e.g., through ether or ester-type linkages. Reactions for such linkages are well known.

Another group of conjugates of formula (I) are those in which T is an estrogen or estrogen equivalent or an antiestrogen. Suitable conjugation or association is provided between a vitamin D compound and an estrogen, may occur between the hydroxy groups in the estrogen and the hydroxyl groups in the vitamin D. An exemplary vitamin D-estrogen conjugate is:

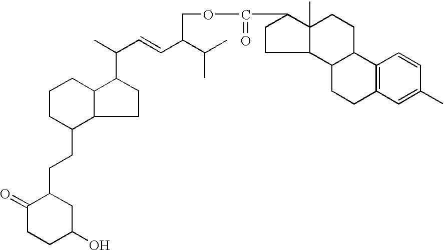

Also desirable among the conjugates of formula (I) are those wherein T is a metal ion, M. Metal ions are known to target many tissue sites, e.g., strontium ion to bone. The conjugates may take the form of direct complexes between a vitamin D moiety and the metal ion wherein the moiety has a negatively charged terminal group, e.g., one having one or more carboxyl groups at C-24, C-25, etc., generally D-X—. The conjugate may be represented by the formula (XVIII):

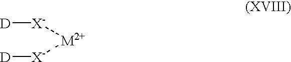

wherein X is a negatively charged group such as a carboxyl, CO₂⁻, and M is a divalent metal ion.

Alternatively, the conjugate may be of the form of formula (V) wherein D and M are associated by a connector, e.g., an amino acid. For example, it has been disclosed that metal ion-amino acid chelates are capable of targeting tissue site delivery. See, e.g., U.S. Pat. Nos. 4,863,898; 4,176,564; and 4,172,072, each of which is incorporated herein by reference. For example, magnesium-lysine chelates have been shown to target bone; and zinc and methionine have been shown to target skin. Such chelates are in addition to the polyacidicamino acid conjugates described hereinbefore and are of the form

(AA)*M*(AA) (XIX)

wherein M is the metal ion and AA is an amino acid residue, (e.g. lysine, arginine, etc.), and * indicates an association of the amino acid residue (AA) with the metal ion (M). In the conjugates in accordance with the present invention, the amino acid of the metal-amino acid chelate is linked to the vitamin D moiety through the amide-type linkage shown above in the case of when T is bisphosphonate. The conjugates are represented by the formula (XX):

D-(AA)*M (XX)

wherein D is a vitamin D moiety having a group capable of forming a linkage to an amino acid, e.g., a hydroxy group; (AA) is the amino acid residue, and M is a metal ion, suitably a divalent ion such as Sr²⁺, Zn²⁺, Mg²⁺, Fe², Cu²⁺, Mn²⁺, Ca²⁺, Cu²⁺, Co²⁺, Cr²⁺ or Mo²⁺. More specifically, conjugates are provided in the form represented by the formula (XXI):

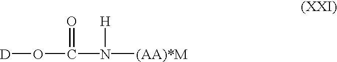

wherein D, M, AA, and * are as defined above.

In another illustrated embodiment, the conjugates of formula (I) are those wherein T is an antibody. Antibodies or antibody fragment which may be used in such conjugates can be prepared by techniques well known in the art. An example of suitable antibodies are immunoglobins, such as IgG, IgA, IgD and IgE. High specificity monoclonal antibodies can be produced by hybridization techniques well known in the art. See, e.g., Kohler et al., 245 Nature 495 (1975); and 6 Eur. J. Immunol. 511 (1976), both of which are incorporated herein by reference. Such antibodies normally may have a highly specific reactivity. Polyclonal antibodies are also suitable for use as the targeting molecule component of the conjugate. However, when the targeting moiety is an antibody, it is most suitably a monoclonal antibody (Mab).

Selected monoclonal antibodies are highly specific for a single epitope, making monoclonal antibodies particularly useful as the targeting molecule components of the conjugates of this invention. Conjugates of vitamin D and monoclonal antibodies can be targeted to specific sites within a target region, such as specific cell-surface antigens of cancerous tissue or in bone tissue. Methods for isolating and producing monoclonal or polyclonal antibodies to specific antigens, such as making antibodies to selected target tissue or even to specific target proteins are known. See, e.g., Molecular Cloning, 2nd ed., Sambrook et al., eds., Cold Spring Harbor Lab. Press, 1989, §18.3 et seq. Polyclonal antibodies can be produced more cheaply than monoclonal antibodies, but polyclonal antibodies inherently are less specific targeting molecules. Nonetheless, it is possible to produce large amounts of monoclonal antibodies suitable for use in the conjugates of the present invention by tissue culture (e.g., a hybridoma cell line).

Conjugates in accordance with the present invention produced using such antibody targeting molecules can be directed against, e.g., cells, organs, tumors, differentiation and other cell membrane antigens, polynucleic acids such as DNA and RNA or any biologically active molecule. Antibodies to receptors of target cells of interest may be suitable for use in the conjugates of this invention.

For those conjugates in accordance with the present invention, when T is a monoclonal antibody, the T*D association is suitably accomplished via a connector “G”, e.g., a biotin-avidin linkage represented by G′-G″, using biotin-avidin methodologies known in the art. Such a conjugate based on a connector, e.g., vitamin D-biotin-avidin-antibody, is suitably represented as

[D-G′]*[G″-T] (XXII)

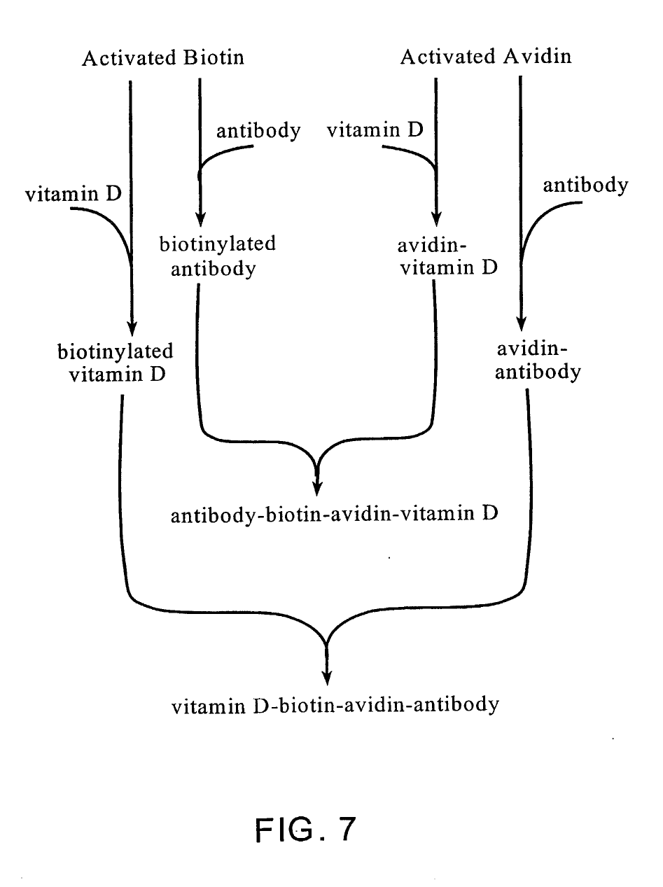

A schematic diagram for the coupling of antibodies to vitamin D compounds or analogs using, e.g., biotin-avidin conjugates, is given in FIG. 7.

Referring to FIG. 7, avidin possesses a high affinity for the coenzyme biotin. This is a strong, noncovalent interaction which has been exploited for the conjugation of antibodies to various compounds. The biotin or avidin is suitably coupled to either the vitamin D compound or the antibody component. As such, a number of different schemes are possible for linking vitamin D compounds and antibodies. For example, biotin is suitably linked to the antibody to form a biotinylated antibody complex, while the avidin is suitably linked to the vitamin D compound to form an avidin vitamin D complex. The two complexes are subsequently reacted to form an antibody-biotin-avidin-vitamin D conjugate. A vitamin D-biotin-avidin-antibody conjugate is formed in a similar manner as shown in FIG. 7.

It may also be desirable to conjugate hormones or other agents (designated as “A”) to the conjugates of formula (I), to form a bifunctional conjugate represented by formula (XXIII)

(D)ₘ*(T)ₙ*(A)ₚ (XXIII)

wherein A represents a therapeutic agent other than vitamin D and p is an integer of 1 or greater, D, T, m and n are as previously defined herein, with the proviso that D and A maintain their biological effectiveness, and * indicates that the targeting molecule is associated with both the vitamin D moiety and with the therapeutic agent other than vitamin D. For example, a bifunctional conjugate of formula (XXIII) is one that has the ability to deliver vitamin D to bone as well as another osteogenic agent such as an estrogen.

Thus, included within the scope of the present invention are conjugates of formula (XXIII) which are bone-therapeutic conjugates wherein A is a hormone or other agent which is known to ameliorate bone diseases or disorders. Such bone agents may include conjugated estrogens or their equivalents, antiestrogens, calcitonin, bisphosphonates, calcium supplements, cobalamin, pertussis toxin, boron, DHEA and other bone growth factors such as transforming growth factor beta, activin or bone morphogenic protein.

Also provided in the present invention are conjugates of formula (XXIII) which are antiproliferative conjugates wherein A is a cytotoxic agent. Cytotoxic agents include antimetabolites, antimicrotabule agents, alkylating agents, platinum agents, anthracyclines, topisomerase inhibitors (e.g., toposide,), antibiotics and other agents such as hormones and antagonists. The antimetabolites include pyrimidine and purine analogs and inhibitors such as 5-fluorouracil, flaxuridine, cytarabine mercaptopurine, thioguanine, pentostatin, cladribine and fludorabine, and folic acid analogs such as meltotrexate. The antimicrotuble agents include vincrestine, vinblastine and taxanes such as paclitaxel and docetaxel. The alkylating agents include nitrogen mustards such as mechlorethamine, cyclophosphamide, ifasfamide, melphalan, chlorambucil, alkyl sulfonates such as busalfan, nitrosoureas such as carmustine and streptozocin and other agents such as hexamethylamine, thiotepa, docarbozine and temozoline. The platinum agents include cisplatin, carboplatin, oxaliplatin, JM-216, and CI-973. The anthracylines include doxorubicin and dounorabicin. The topoisomerase inhibitors include etoposide, teniposide, the camptothecens such as toptecan and irinotecan. The antibiotics include mitomycin, andriamycin, dactinomycin, daunomycin, idarabicin and bleomycin. Other chemotherapeutic agents include hormones such as adrenocorticosteroids (e.g. prednisone), progestins (e.g., hydroxyprogestetone caproate, medroxyprogesterone acetate, megestrol acetate), estrogens (e.g., diethylstibestrol, ethinyl estrodiol), antiestrogens (e.g., tamoxifen, anastrozole), androges (e.g., testosterone propionate, fluoxymesterone), antiandgrogen (e.g., flutamide) and gonado-releasing hormone analogs (e.g., leuprolide). Still other agents useful in neoplastic disease are biological response modifiers (e.g., interferon-alpha interleuben-2), anthracenediones (e.g., mitoanthrone), substituted ureas (e.g. hydroxyurea), methylhydrozine derivatives (e.g. procarbazine) andrenocorticol suppressants (e.g. mitotane, aminoglutethimide), tyrosim kinase inhibitors (e.g. einatinib) enzyme (e.g. 1-asparogine), and estramustine phosphate, and prednimustine.

Included within the scope of the present invention are all possible enantiomers of any conjugate of the invention which exhibits optical isomerism, all possible geometric isomers due to a cis-trans configuration at double bonds and epimers where differing stereo-specificity is possible. Additionally, all pharmaceutically acceptable salts of compounds described herein, such as sodium, potassium, lithium, ammonium salts of the compounds, and the like.

The magnitude of a prophylactic or therapeutic dose of the conjugates in accordance with the present invention will vary with the nature or the severity of the condition to be treated and with the particular composition and its route of administration. In general, the daily dose range for use in bone diseases lies within the range of about 0.025 mmol/kg of body weight to about 2.5 mmol/kg. The daily dose for treatment of hyperproliferative diseases, such as cancers, is in the range of about 0.025 mmol/kg to about 5 mmol/kg of body weight. The conjuates in accordance with the present invention can be given in daily dose or episodic dose, e.g., once every 2-6 days or once a week, the dose in each day can be a single dose or divided into 2-4 subdoses which can be given, e.g., an hour apart until the total dose is given.

Small acidic peptide conjugates of formula (I) are particularly useful as active compounds in pharmaceutical compositions having reduced side effects and low toxicity as compared with the known analogs of active forms of vitamin D₃.

The pharmacologically active conjugates of this invention can be processed in accordance with conventional methods of pharmacy to produce medicinal agents for administration to patients, e.g., mammals including humans. Any suitable route of administration may be employed for providing an effective dosage of the conjugate. For example, oral, rectal, topical, parenteral, intravenous, intramuscular, subcutaneous, ocular, nasal, buccal, and the like may be employed.

Pharmaceutical compositions of the present invention include the conjugate of the present invention as an active ingredient or a pharmaceutically acceptable salt thereof, and may also contain a pharmaceutically acceptable carrier and optionally other therapeutic ingredients. The compositions are those suitable for the various routes of administration described herein, although the most suitable route in any given case will depend on the nature and severity of the condition being treated and on the nature of the active ingredient conjugate. They are conveniently presented in unit dosage form.

Suitable pharmaceutically acceptable carriers for use in the composition or method of the present invention include, but are not limited to water, salt solutions, alcohols, gum arabic, vegetable oils (e.g., corn oil, cottonseed oil, peanut oil, olive oil, coconut oil), fish liver oils, oily esters such as Polysorbate 80, polyethylene glycols, gelatine, carbohydrates (e.g., lactose, amylose or starch), magnesium stearate, talc, silicic acid, viscous paraffin, fatty acid monoglycerides and diglycerides, pentaerythritol fatty acid esters, hydroxy methylcellulose, polyvinyl pyrrolidone, etc.

The pharmaceutical preparations can be sterilized and, if desired, be mixed with auxiliary agents, e.g., lubricants, preservatives, stabilizers, wetting agents, emulsifiers, salts for influencing osmotic pressure, buffers, coloring and flavoring. Additionally, the conjugate of the present invention may be coated or shielded to prevent immunogenicity or reticuloandothelial (RES) response by, e.g., the liver. Agents which can be used for this purpose include polyethylene glycol (PEG) and others known in the art.

In another aspect, the present invention is a method of site-specific delivery of a vitamin D moiety to a tissue of interest in a patient. The method includes: (1) providing a conjugate of a vitamin D moiety and at least one targeting molecule moiety in a pharmaceutically acceptable carrier; and (2) administering a therapeutically effective dose of the conjugate of the present invention described herein. The delivery of vitamin D to a specific site (i.e., tissue of interest) provides for site-specific methods of treatment of significant diseases and pathological conditions utilizing vitamin D. Accordingly, in another aspect, the present invention provides a method of inhibiting hyperproliferation of malignant or neoplastic cells in a tissue of interest. The method includes treating the cells with an antiproliferative amount of a vitamin D conjugate. The conjagate includes a vitamin D moiety and a target moiety having affinity for a tissue of interest; the cells suitably have a vitamin D receptor. The cells may be cancers of the breast, colon, lung, neck and head, endometrium, bladder, cervix, testes, prostate, ovaries, liver, squamous cell carcinoma, myloid and lymphocytic leukemia including acute lymphoblastic leukemia, acute myelogenous leukemia, chronic myelogenous leukemia, chronic lymphocytic leukemiaa as well as myeleoplastic syndromes, lymphoma, medullary thyroid carcinoma, melanoma, multiple myeloma, retinoblastoma, sarcomas of soft tissue and bone, or other hyperproliferative disease states such as psoriasis.

Further, in the treatment of hyperproliferative diseases, conjugates in accordance with the present invention are suitably those that also include a cytotoxic agent in addition to the vitamin D moiety, e.g., the conjugates represented by formula (XXIII). The conjugate is preferably delivered in one of the following forms, depending upon the specific method of administration.

For parenteral application, particularly suitable are injectable, sterile solutions, preferably oily or aqueous solution, as well as suspensions, emulsions, or implants, including suppositories. Ampoules are convenient unit dosages.

For enteral application, particularly suitable are tablets, dragees, liquids, drops, suppositories, lozenges, powders, or capsules. A syrup, elixir, or the like can be used if a sweetened vehicle is desired. Because of their ease of administration, tablets and capsules represent the most advantageous oral dosage unit.

For rectal administration, conjugates are formed into a pharmaceutical composition containing a suppository base such as cacao oil or other triglycerides. To prolong storage life, the composition advantageously includes an antioxidant, such as ascorbic acid, butylated hydroxyanisole or hydroquinone.

Suitable topical formulations include transdermal devices, aerosols, creams, ointments, lotions, dusting powders and the like.

Depending on the nature of the conjugate, oral administration of the pharmaceutical compositions of the present invention is suitable. A daily dosage of the compounds according to this invention generally is about 0.025 to about 2.5 mmol/kg of weight of the subject, suitably about 0.025 to about 1 mmol/kg. Generally, the conjugates of this invention are dispensed by unit dosage form in a pharmaceutically acceptable carrier. For treatment of hyperproliferative diseases such as cancers, the enteral dosage of the conjugates of formula (I), is suitably about 1 mmol to about 100 mmol per unit dosage; for bone diseases, about 0.5 mmol to 50 mmol per unit dosage. Parenteral administration is also suitable with appropriate adjustment of doses given herein.

In addition, those skilled in the art will also appreciate that such dosages may be encapsulated in time release, e.g., sustained, delayed or controlled release delivery systems such as a liposome delivery system, polysaccharides exhibiting a slow release mechanism, salistic or other polymer implants or microspheres, as well as those where the active ingredient is suitably protected with one or more differentially degradable coatings, e.g., by microencapsulation, enteric coating, multiple coatings, etc., and such means effect continual dosing of compositions contained therein. For example, an enteric coating is suitably one which is resistant to disintegration in gastric juice. It may also be possible to freeze-dry the active ingredient conjugate and use the lyophilizate obtained, e.g., for the preparation of products for injection.

It will further be appreciated that the actual preferred amounts of active compound in a specific case will vary according to the efficacy of the specific compound employed, the particular compositions formulated, the mode of application, and the particular situs and organism being treated. For example, the specific dose for a particular patient depends on age, sex, body weight, general state of health, on diet, on the timing and mode of administration, on the rate of excretion, and on medicaments used in combination and the severity of the particular disorder to which the therapy is applied. Dosages for a given host can be determined using conventional considerations, e.g., by customary comparison of the differential activities of the subject compounds and of a known agent, such as by means of an appropriate conventional pharmacological protocol. The effective amount of the conjugate of the present invention may provide an amount of active compound which is lower than the effective amount of the active compound when administered alone.

The present invention is further explained by the following examples which should not be construed by way of limiting the scope of the present invention.

### EXAMPLE 1

### Synthesis of 1α-(OH)-24-aminoalkyl-1,1-bisphosphonate-D₂ conjugate (7)

Reference is made to the reaction scheme of FIG. 1. A solution of the known alcohol (1) (1.0 g, 1.52 mmol) in toluene (5 mL) is added to 22 mL of a 12.5% solution of phosgene in toluene and the solution is stirred at room temperature for 20 hrs. The reaction mixture is concentrated under reduced pressure to provide the chloroformate (2).

Pyridine (0.18 mL, 2.2 mmol) is added to a solution of (2) (1.1 g, 1.53 mmol) and tetraisopropyl 4-aminobutyl-1,1-bisphosphonate 3 (see, Saari et al., U.S. Pat. No. 5,183,815) (0.92 g, 2.2 mmol) in CH₂Cl₂ (12 mL), and the mixture is stirred at room temperature for three days. The reaction mixture is concentrated under reduced pressure, and the residue is purified by flash chromatography over silica gel, utilizing methanol/chloroform as the eluent, to yield compound (4).

A solution of compound (4) (0.80 g, 0.74 mmol) in tetrahydrofuran (THF) (10 mL) and tetrabutylammonium fluoride (TBAF) (2.2 mL of a 1.0 M THF solution, 2.2 mmol) is stirred at room temperature for 24 hrs. The reaction mixture is diluted with water (30 mL) and extracted with CH₂Cl₂ (3×40 mL). The combined CH₂Cl₂ fractions are dried over anhydrous sodium sulfate, filtered, and the filtrate is concentrated under reduced pressure. The residue is purified by flash chromatography over silica gel, utilizing methanol/chloroform as the eluent, to yield tetraisopropyl ester compound (5).

Trimethylsilylbromide (0.39 mL, 2.92 mmol) is added to a solution of the tetraisopropyl ester compound (5) (0.5 g, 0.58 mmol) in CH₂Cl₂ (6 mL), and the mixture is stirred at room temperature for 24 hrs. under an inert atmosphere. The reaction mixture is concentrated under reduced pressure and the residue is diluted with water (15 mL). The mixture is filtered and the filtrate lyophilized to provide compound (6).

A solution of compound (6) (250 mg, 0.36 mmol) and 9-acetylanthracene (21 mg) in methanol (93 mL) is placed in an ACE 500-mL photo reactor and the solution is purged with nitrogen for 15 min. The reaction mixture is cooled to 0° C. and irradiated with a 400 W Hanovia lamp filtered through uranyl glass for 2 hrs. The reaction mixture is filtered, and the filtrate is concentrated under reduced pressure. The residue is diluted with water (10 mL). The mixture is filtered and the filtrate lyophilized to provide the above-titled conjugate (7).

### EXAMPLE 2

### Synthesis of 1-aminoalkyl-1,1-bisphosphonate-24-(OH)-D₂ (16)

Reference is made to the reaction scheme depicted in FIGS. 2A & 2B. To a 0° C. solution of compound (1) (2.0 g, 3.04 mmol), and N,N-diisopropylethylamine (0.78 g, 6.1 mmol) in CH₂Cl₂ (25 mL) is added chloromethyl methyl ether (0.29 g, 3.6 mmol). The resulting reaction mixture is stirred at 0° C. for 1 hr., then at room temperature for 7 hrs, prior to dilution with water (30 mL). The separated aqueous phase is extracted with CH₂Cl₂ (3×25 mL), and the combined organic phases are dried over anhydrous sodium sulfate, filtered, and the filtrate is concentrated in vacuo. The residue is purified by flash chromatography over silica gel to provide compound (8).

A solution of compound (8) (1.99 g, 2.84 mmol) in THF (38 mL) and TBAF (8.4 ML of a 1.0 M THF) is stirred at room temperature for 24 hrs. The reaction mixture is diluted with water (100 mL), and extracted with CH₂Cl₂ (3×100 mL). The combined CH₂Cl₂ phases are dried over anhydrous sodium sulfate, filtered, and the filtrate is concentrated under reduced pressure. The residue is purified by flash chromatography over silica gel, utilizing methanol/chloroform as the eluent, to yield the diol compound (9).

To a solution of diol (9) (1.25 g, 2.64 mmol), N,N-dimethylformamide (20 mL), and imidazole (0.54 g, 7.93 mmol) is added tert-butyldimethylsilyl chloride (0.40 g, 2.64 mmol). The reaction mixture is stirred at room temperature for 6 hrs., prior to dilution with water (60 mL) and extraction with CH₂Cl₂ (3×70 μL). The combined organic phases are washed with brine (50 mL), dried over anhydrous sodium sulfate, filtered, and the filtrate is concentrated under reduced pressure. The residue is purified by flash chromatography over silica gel, to provide two products separately. They are identified in FIG. 2A as alcohol (10) and alcohol (11).

As illustrated at the top of FIG. 2B, a solution of alcohol (10) (0.5 g, 0.85 mmol) in toluene (2.5 mL) is added to 12.3 mL of a 12.5% solution of phosgene in toluene and the solution is stirred at room temperature for 20 hrs. The reaction mixture is concentrated under reduced pressure to provide chloroformate (12).

Pyridine (0.09 mL, 1.1 mmol) is added to a solution of cloroformate (12) (0.5 g, 0.77 mmol) and tetraisopropyl 4-aminobutyl-1,1-bisphosphonate (3) (0.46 g, 1.1 mmol) in CH₂Cl₂ (6 mL) and the mixture is stirred at room temperature for three days. The reaction mixture is concentrated under reduced pressure, and the residue is purified by flash chromatography over silica gel, utilizing methanol/chloroform as the eluent, to yield compound (13).

A solution of compound (13) (0.50 g, 0.49 mmol) in THF (7 mL) and TBAF (0.74 nL of a 1.0 M THF solution, 0.74 mmol) is stirred at room temperature for 24 hrs. The reaction mixture is diluted with water (20 mL) and extracted with CH₂Cl₂ (3×30 mL). The combined CH₂Cl₂ fractions are dried over anhydrous sodium sulfate, filtered, and the filtrate is concentrated under reduced pressure. The residue is purified by flash chromatography over silica gel, utilizing methanol/chloroform as the eluent, to provide compound (14).

Trimethylsilylbromide (0.26 mL, 1.96 mmol) is added to a solution of tetraisopropyl ester (14) (0.35 g, 0.39 mmol) in CH₂Cl₂ (4 mL) and the mixture is stirred at room temperature for 24 hrs. under an inert atmosphere. The reaction mixture is concentrated under reduced pressure and the residue is diluted with water (15 mL) and methanol (3 mL) and is stirred for 8 hrs. The mixture is filtered and the filtrate lyophilized to provide compound (15).

A solution of compound (15) (0.21 g, 0.31 mmol) and 9-acetylanthracene (18 mg) in methanol (80 mL) is placed in an ACE 500-mL photo reactor, and the solution is purged with nitrogen for 15 min. The reaction mixture is cooled to 0° C. and is irradiated with a 400 W Hanovia lamp filtered through uranyl glass for 2 hrs. The reaction mixture is filtered, and the filtrate concentrated under reduced pressure. The residue is diluted with water (10 mL). The mixture is filtered and the filtrate lyophilized to provide the above-titled conjugate (16).

### EXAMPLE 3

### Synthesis of 1α,24-(OH)₂-3-aminoalkyl-1,1-bisphosphonate-D₂ (21)

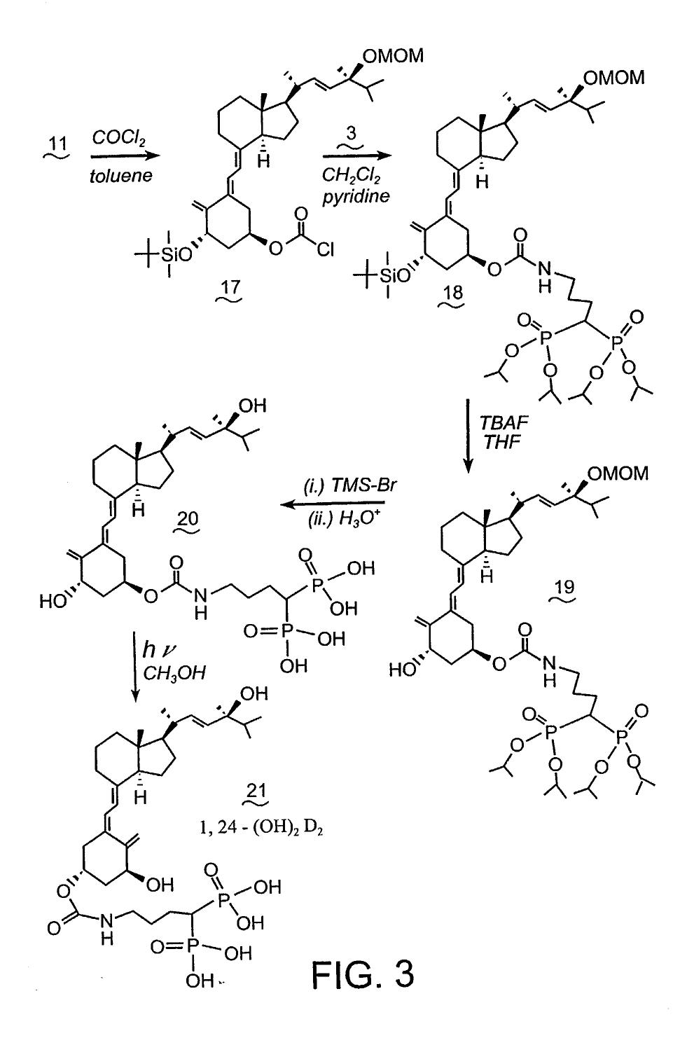

Reference is made to FIG. 3. A solution of alcohol (11) (0.5 g, 0.85 mmol) in toluene (2.5 mL) is added to 12.3 mL of a 12.5% solution of phosgene in toluene and the solution is stirred at room temperature for 20 hrs. The reaction mixture is concentrated under reduced pressure to provide chloroformate (17).

Pyridine (0.081 mL, 1 mmol) is added to a solution of (17) (0.45 g, 0.69 mmol) and tetraisopropyl 4-aminobutyl-1,1-bisphosphonate (0.41 g, 1 mmol) in CH₂Cl₂ (5 mL), and the mixture is stirred at room temperature for three days. The reaction mixture is concentrated under reduced pressure, and the residue is purified by flash chromatography over silica gel, utilizing methanol/chloroform as the eluent, to provide compound (18).

A solution of compound (18) (0.47 g, 0.46 mmol) in THF (6.5 mL) and TBAF (0.7 mL of a 1.0 M THF solution, 0.7 mmol) is stirred at room temperature for 24 hrs. The reaction mixture is diluted with water (20 mL) and extracted with CH₂Cl₂ (3×30 mL). The combined CH₂Cl₂ fractions are dried over anhydrous sodium sulfate, filtered, and the filtrate is concentrated under reduced pressure. The residue is purified by flash chromatography over silica gel, utilizing methanol/chloroform as the eluent, to yield compound (19).

Trimethylsilylbromide (0.23 mL, 1.74 mmol) is added to a solution of tetraisopropyl ester (19) (0.31 g, 0.34 mmol) in CH₂Cl₂ (4 mL) and the mixture is stirred at room temperature for 24 hours under an inert atmosphere. The reaction mixture is concentrated under reduced pressure, and the residue is diluted with water (15 mL) and methanol (3 mL), and stirred for 8 hours. The mixture is filtered, and the filtrate is lyophilized to provide compound (20).

A solution of compound (20) (0.21 g, 0.31 mmol) and 9-acetylanthracene (18 mg) in methanol (80 mL) is placed in an ACE 500-mL photo reactor, and the solution is purged with nitrogen for 15 min. The reaction mixture is cooled to 0° C. and irradiated with a 400-W Hanovia lamp filtered through uranyl glass for 2 hours. The reaction mixture is filtered, and the filtrate is concentrated under reduced pressure. The residue is diluted with water (10 mL). The mixture is filtered, and the filtrate is lyophilized to provide the above-titled conjugate (21).

### EXAMPLE 4

### Synthesis of 1α-aminoalkyl-1,1-bisphosphonate-25-(OH)-D₃ (32)

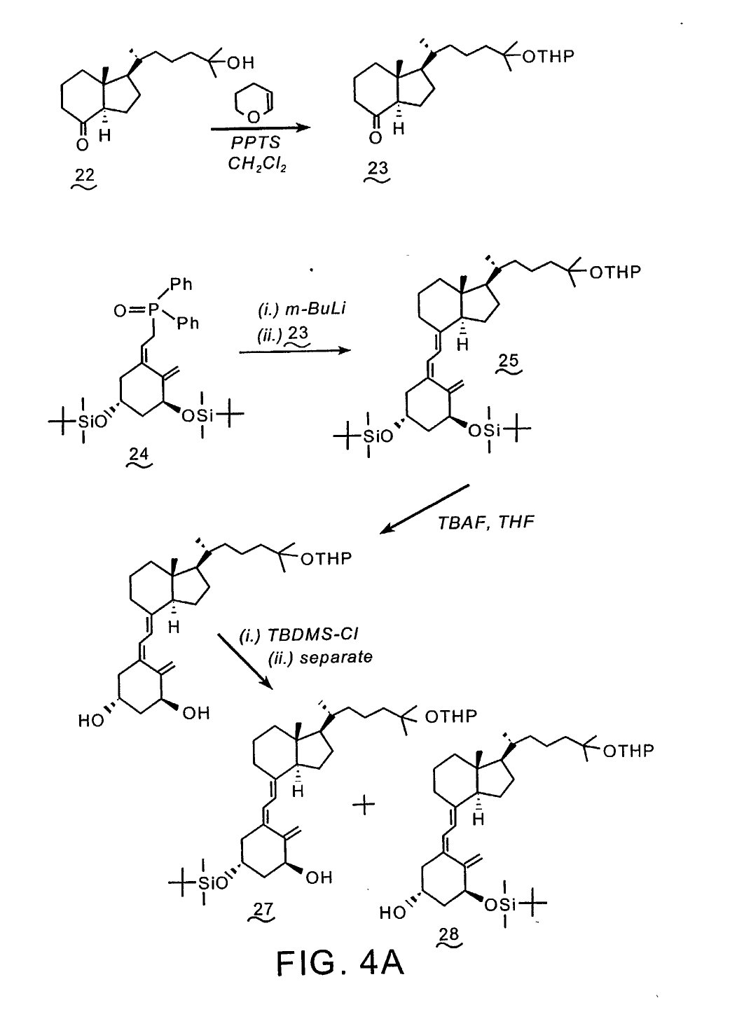

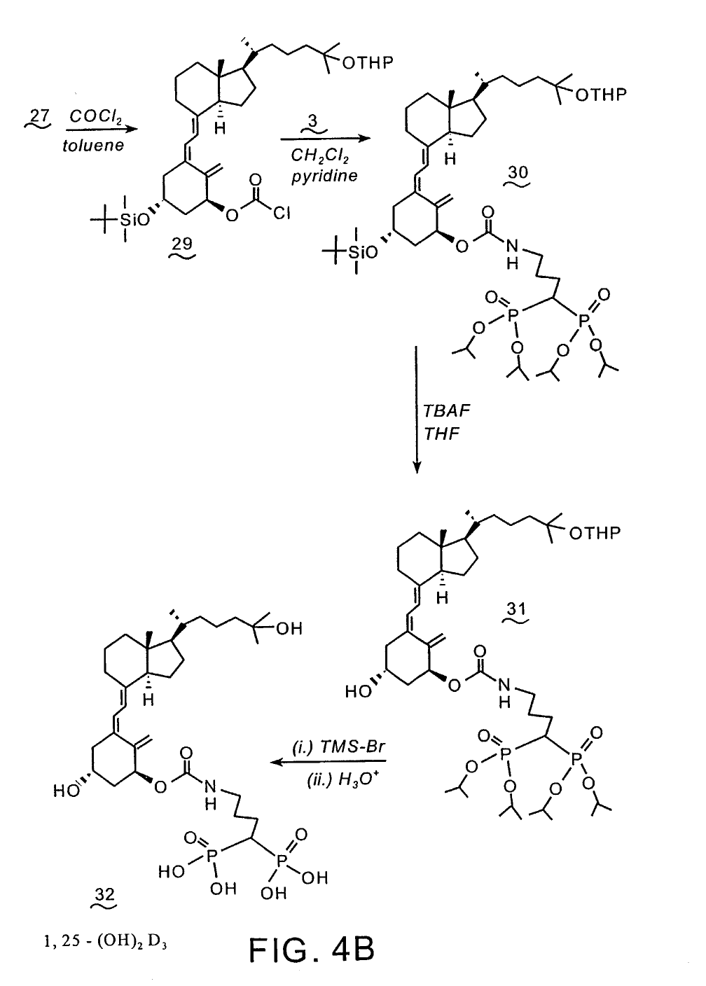

Reference is made to the reaction scheme depicted in FIGS. 4A and 4B. A solution of the known ketone (22) (3.1 g, 11.1 mmol) (see, Baggiolini et al., 51 J. Org. Chem. 3098 (1986)), incorporated herein by reference, 3,4-dihydro-2H-pyran (1.52 mL, 16.7 mmol), and pyridinium p-toluenesulfonate (0.1 g, 0.4 mmol) are dissolved in CH₂Cl₂ (50 mL), and are stirred at room temperature for 24 hrs. The reaction mixture is washed with water (30 mL), and the organic phase is dried over anhydrous sodium sulfate, filtered, and concentrated under reduced pressure. The residue is purified by flash chromatography over silica gel to yield ketone compound (23).

A solution of the known phosphine oxide (24) (1.35 g, 2.32 mmol) in 35 mL of anhydrous THF is cooled to −78° C. and treated with m-butyllithium (1.45 mL of a 1.6 M solution in hexane) dropwise. The anion solution is stirred for 5 min. at −78° C. prior to the addition of ketone (23) (0.57 g, 1.56 mmol) dissolved in anhydrous THF (10 mL) during 10-15 min. The reaction mixture is stirred for 2 hrs at −78° C. then diluted with 2 N sodium potassium tartrate (6 mL) and 2 N potassium bicarbonate (6 mL). The solution is warmed to room temperature and extracted with ethyl acetate (4×25 mL). The combined organic fractions are washed with brine (30 mL), dried over anhydrous sodium sulfate, filtered, and concentrated under reduced pressure. The residue is purified by flash chromatography over silica gel, utilizing ethyl acetate/hexane as eluent, to provide compound (25).

A solution of compound (25) (0.95 g, 1.3 mmol) in THF (17 mL) and TBAF (3.9 mL of a 1.0 M THF solution, 3.9 mmol) is stirred at room temperature for 24 hrs. The reaction mixture is diluted with water (50 mL) and extracted with CH₂Cl₂ (3×60 mL). The combined CH₂Cl₂ fractions are dried over anhydrous sodium sulfate, filtered, and the filtrate concentrated under reduced pressure. The residue is purified by flash chromatography over silica gel, utilizing methanol/chloroform as eluent, to provide diol compound (26).

To a solution of the diol compound (26) (0.55 g, 1.1 mmol), N, N-dimethylformamide (8 mL), and imidazole (0.255 g, 3.3 mmol) is added tert-butyldimethylsilyl chloride (0.166 g, 1.1 mmol). The reaction mixture is stirred at room temperature for 6 hrs., prior to dilution with water (30 mL) and extraction with CH₂Cl₂ (3×40 mL). The combined organic phases are washed with brine (30 mL), dried over anhydrous sodium sulfate, filtered, and concentrated under reduced pressure. The residue is purified by flash chromatography over silica gel, to provide two separate products. They are identified, at the bottom of FIG. 4A, as alcohol (27) and alcohol (28).

A solution of alcohol (27) (0.25 g, 0.41 mmol) in toluene (3 mL) is added to 6 mL of a 12.5% solution of phosgene in toluene, and the reaction mixture is stirred at room temperature for 20 hrs. The reaction mixture is concentrated under reduced pressure to provide chloroformate (29), as shown at the top of FIG. 4B.

Pyridine (0.047 mL, 0.57 mmol) is added to a solution of (29) (0.27 g, 0.4 mmol) and tetraisopropyl 4-aminobutyl-1,1-bisphosphonate (0.24 g, 0.57 mmol) in CH₂Cl₂ (4 mL) and the mixture is stirred at room temperature for three days. The reaction mixture is concentrated under reduced pressure, and the residue is purified by flash chromatography over silica gel, utilizing methanol/chloroform as eluent, to provide compound (30).

A solution of compound (30) (0.36 g, 0.35 mmol) in THF (5 mL) and TBAF (0.52 mL of a 1.0 M THF solution, 0.52 mmol) is stirred at room temperature for 24 hrs. The reaction mixture is diluted with water (15 mL) and extracted with CH₂Cl₂ (3×25 mL). The combined CH₂Cl₂ fractions are dried over anhydrous sodium sulfate, filtered, and concentrated under reduced pressure. The residue is purified by flash chromatography over silica gel, utilizing methanol/chloroform as eluent, to provide compound (31).

Trimethylsilylbromide (0.20 mL, 1.54 mmol) is added to a solution of tetraisopropyl ester (31) (0.28 g, 0.30 mmol) in CH₂Cl₂ (4 mL), and the mixture is stirred at room temperature for 24 hrs. under an inert atmosphere. The reaction mixture is concentrated under reduced pressure and the residue diluted with water (15 mL) and methanol (3 mL) and stirred for 0.5 hr. The mixture is filtered, and the filtrate is lyophilized to provide the above-titled conjugate (32).

### EXAMPLE 5

### Synthesis of 1α,25-(OH)₂-3-aminoalkyl-1,1 bisphosphonate-D₃ (36)

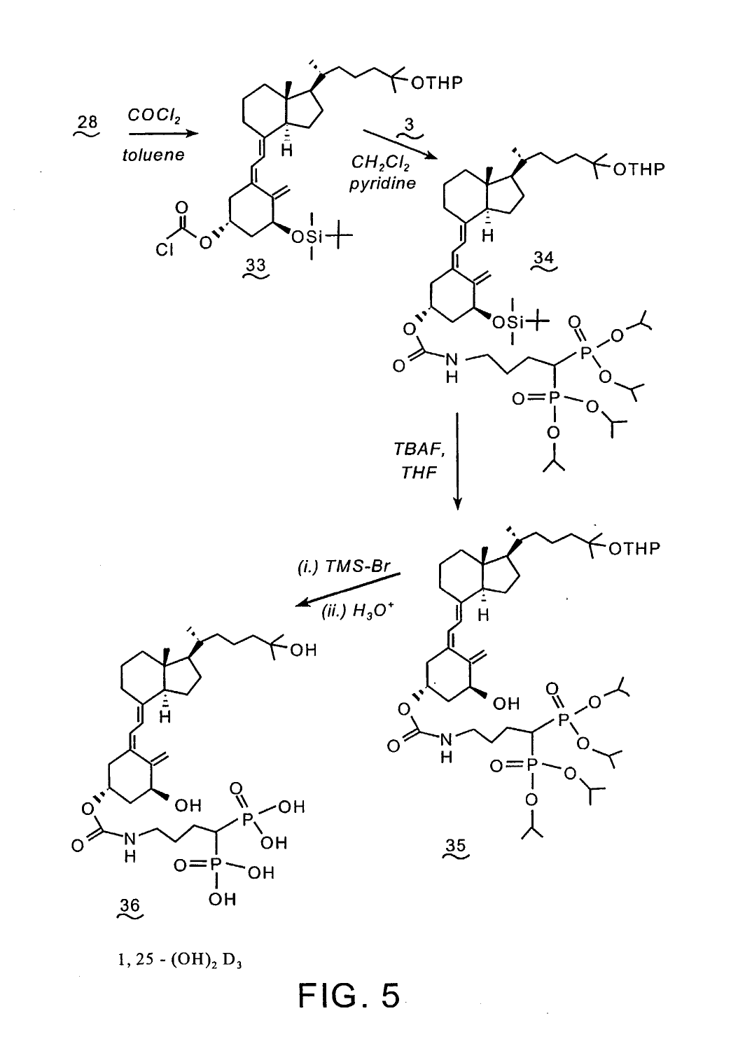

Reference is made to FIG. 5. A solution of alcohol (28) (0.26 g, 0.43 mmol) in toluene (3 mL) is added to 6.2 mL of a 12.5% solution of phosgene in toluene and the reaction is stirred at room temperature for 20 hrs. The reaction mixture is concentrated under reduced pressure to provide chloroformate (33).

Pyridine (0.049 mL, 0.59 mmol) is added to a solution of chloroformate (33) (0.28 g, 0.42 mmol) and tetraisopropyl 4-aminobutyl-1,1-bisphosphonate (0.25 g, 0.59 mmol) in CH₂Cl₂ (5 mL), and the mixture is stirred at room temperature for three days. The reaction mixture is concentrated under reduced pressure, and the residue is purified by flash chromatography over silica gel, utilizing methanol/chloroform as eluent, to yield compound (34).

A solution of compound (34) (0.37 g, 0.36 mmol) in THF (5 mL) and TBAF (0.54 mL of a 1.0 M THF solution, 0.54 mmol) is stirred at room temperature for 24 hrs. The reaction mixture is diluted with water (15 mL) and extracted with CH₂Cl₂ (3×25 m]L). The combined CH₂Cl₂ fractions are dried over anhydrous sodium sulfate, filtered, and the filtrate concentrated under reduced pressure. The residue is purified by flash chromatography over silica gel, utilizing methanol/chloroform as eluent, to yield tetraisopropyl ester (35).

Trimethylisilylbromide (0.21 mL, 1.60 mmol) is added to a solution of the tetraisopropyl ester (35) (0.29 g, 0.31 mmol) in CH₂Cl₂ (5 mL), and the mixture is stirred at room temperature for 24 hrs under an inert atmosphere. The reaction mixture is concentrated under reduced pressure, and the residue is diluted with water (15 mL) and methanol (3 mL) and stirred for 0.5 hr. The mixture is filtered, and the filtrate is lyophilized to provide the above-titled conjugate (36).

### EXAMPLE 6

### Synthesis of 1-(OH)-25-aminoalkyl-1,1-bisphosphonate-D₃ (41)

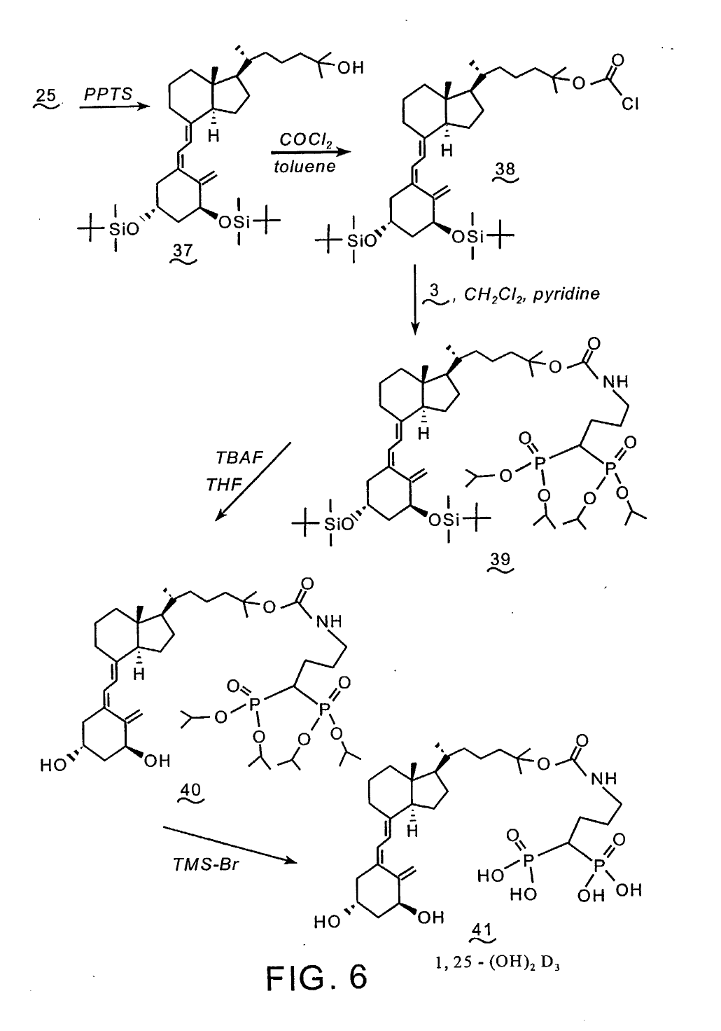

Reference is made to FIG. 6. To a solution of ether (25) (1.31 g, 1.8 mmol) in methanol (10 mL) and water (2 mL) is added pyridinium p-toluenesulfonate hydrate (0.034 g, 0.18 mmol). The reaction mixture is stirred at room temperature for 1 hr, diluted with water (20 mL) and extracted with CH₂Cl₂ (3×30 mL). The combined organic phases are dried over anhydrous sodium sulfate, filtered, and concentrated in vacuo. The residue is purified by flash chromatography over silica gel, utilizing methanol/chloroform as eluent, to provide alcohol (37).

A solution of alcohol (37) (1.09 g, 1.7 mmol) in toluene (9 mL) is added to 25 mL of a 12.5% solution of phosgene in toluene, and the reaction is stirred at room temperature for 20 hrs. The reaction mixture is concentrated under reduced pressure to provide chloroformate (38).

Pyridine (0.19 mL, 2.32 mmol) is added to a solution of cloroformate (38) (1.17 g, 1.65 mmol) and tetraisopropyl 4-aminobutyl-1,1-bisphosphonate (0.98 g, 2.32 mmol) in CH₂Cl₂ (20 mL), and the mixture is stirred at room temperature for three days. The reaction mixture is concentrated under reduced pressure, and the residue is purified by flash chromatography over silica gel, utilizing methanol/chloroform as eluent, to yield alcohol (39).

A solution of alcohol (39) (1.61 g, 1.5 mmol) in THF (20 mL) and TBAF (4.5 mL of a 1.0 M THF solution, 4.5 mmol) is stirred at room temperature for 24 hrs. The reaction mixture is diluted with water (60 mL) and extracted with CH₂Cl₂ (3×60 mL). The combined CH₂Cl₂ phases are dried over anhydrous sodium sulfate, filtered, and the filtrate is concentrated under reduced pressure. The residue is purified by flash chromatography over silica gel, utilizing methanol/chloroform as eluent, to provide tetraisopropyl ester compound (40).

Trimethylsilylbromide (0.75 mL, 5.68 mmol) is added to a solution of the tetraisopropyl ester compound (40) (0.93 g, 1.1 mmol) in CH₂Cl₂ (20 mL), and the mixture is stirred at room temperature for 24 hrs under an inert atmosphere. The reaction mixture is concentrated under reduced pressure and the residue is diluted with water (30 mL). The mixture is filtered and the filtrate is lyophilized to provide the above-titled compound (41).

### EXAMPLE 7

### Synthesis of a Vitamin D-Estrogen Conjugate

A vitamin D-17-β-estradiol conjugate is prepared using the same initial step of the scheme of, e.g., FIG. 1 to form a vitamin D-acyl chloride. The acyl chloride is reacted with 17β-estradiol using a similar scheme as illustrated in FIG. 1

### EXAMPLE 8

### Synthesis of Other Vitamin D Conjugates Via an Ester or Amide Linkage

The acyl chloride formed in the first step of the syntheses illustrated in FIGS. 1 and 3 and in the foregoing examples is a reactive intermediate capable of forming an ester linkage with hydroxyl groups or an amide linkage with amino groups. The reactive acyl chloride should be of value in forming such linkages for other vitamin conjugates wherein the target molecule has a hydroxyl or an amino group. Such illustrative target molecules include polyaminoacids, peptides, and metal-amino acid chelates.

In summary, the present invention provides vitamin D conjugates useful in targeting applications. The conjugates include a vitamin D moiety and a targeting moiety having affinity for a tissue of interest. The conjugates are characterized by an ability for site-specific targeting of vitamin D compounds, e.g., a conjugate of vitamin D and a bone affinity agent is designed to transport and deliver vitamin D to bone tissue.

All patents, publications and references cited herein are hereby fully incorporated by reference. In the case of conflict between the present disclosure and the incorporated patents, publications and references, the present disclosure should control.

While the present invention has now been described and exemplified with some specificity, those skilled in the art will appreciate the various modifications, including variations, additions, and omissions, that may be made in what has been described. Accordingly, it is intended that these modifications are to be included within the spirit and purview of this application and the scope of the appended claims.

We claim:

1. A method of inhibiting hyperproliferation of malignant or neoplastic cells, comprising treating the cells with an antiproliferative amount of a vitamin D conjugate, the conjugate comprising a vitamin D moiety and a tumor-seeking moiety.

2. A method as set forth in claim 1, where the cells comprise a vitamin D receptor.

3. A method as set forth in claim 2, wherein the cells are cancers of the breast, colon, lung, neck and head, pancreas, endometrium, bladder, cervix, testes, prostate, skin, ovaries, liver, skin carcinoma, small cell and non-small cell cancer of the lung, squamous cell carcinoma, myeloid and lymphocytic leukemias, lymphoma, medullary thyroid carcinoma, melanoma, multiple myeloma, retinoblastoma or sarcomas of the soft tissues and bone.

4. A method as set forth in claim 3, wherein an amount of the conjugate is administered to a human patient in need thereof which amount is effective to inhibit growth of the cells.

5. A method as set forth in claim 1, wherein the vitamin D moiety includes a moiety of at least one of 1α-dihydroxyvitamin D₂, 1α,2-dihydroxyvitamin D₄, 1α,24-dihydroxyvitamin D₂, 1α,25-dihydroxyvitamin D₃, 1α hydroxyvitamin D₃, 1α,25-dihydroxyvitamin D₂, 1α,25-dihydroxyvitamin D₄, 1α,24,25-dihydroxyvitamin D₂, seocalcitol, calcipotriol, maxacalcitol, falecalcitriol, and paricalcitol.

6. A method as set forth in claim 1, wherein the tumor-seeking moiety includes at least one of antibodies, oxidized glycosylated proteins, polylysine, human serum albumin, dextrans, peptides and proteins having affinity for cellular receptors, polyanionic compounds and polymers, polysulphated compounds and polymers, and peptide analogs.

7. A method as set forth in claim 6, wherein the antibodies includes at least one of antibodies for vascular permeability factor and monoclonal antibodies.

8. A method as set forth in claim 6, wherein the cellular receptors includes at least one of gastrin releasing peptide receptor, epidermal growth factor receptor, platelet-derived growth factor receptor, tumor necrosis factor receptor, fibroblast growth factor receptor, insulin-like growth factor receptor, transfertin receptor, laminin receptor, cytokine receptors, fibronectin receptor, interleukin receptor, interferon receptors, bombesen/gastrin-releasing peptide receptor, and somatostatin receptor.

9 A method as set forth in claim 6, wherein polyanionic compounds and polymers include at least one of sumarin, and analogues and derivatives of sumarin.

10. A method as set forth in claim 6, wherein the polysulphated compounds and polymers include at least one of heparin, heparan sulfate, chrondroitin sulfate, keratan sulfate, dermatan sulfate, sulfated chitin, sulfated chitosan, sulfated alginic acid, pentosan polysulfate, sulfated cyclodextrins, polystyrene sulfonate, sulfated polyvinyl alcohol, polyvinyl sulfate, and polyethylene sulfonate.

11. A method as set forth in claim 6, wherein the peptide analogs include at least one of analogs of LH-RH, dombesin, and somatostatin.

12. A method of treating a human to alleviate the pathological effects of breast cancer, colon cancer, testicular cancer, pancreatic cancer, endometrial cancer, prostate cancer, skin carcinoma, small cell and non-small cell cancer of the lung, squamous cell of the head and neck, bladder, ovarian and cervical cancers, myeloid and lymphocytic leukemias, lymphoma, hepatic tumors, medullary thyroid carcinoma, multiple myeloma, melanoma, retinoblastoma, soft tissue sarcoma, bone sarcoma, or psoriasis comprising administering to the human a therapeutic amount of a vitamin D conjugate, the conjugate comprising a vitamin D moiety and a target molecule moiety.

13. A method as set forth in claim 12, wherein the vitamin D conjugate further comprises a moiety which is a therapeutic agent other than vitamin D.

14. A method as set forth in claim 13, wherein the therapeutic agent is a cytotoxic agent.

15. A method as set forth in claim 14, wherein the cytotoxic agent includes at least one of an antimetabolite, antimicrotubule agent, an alkyating agent, a platinum agent, an anthracycline, a topoisomase inhibitor, an antibiotic, a hormone or a biological response modifier.

16. A method as set forth in claim 15, wherein the antimicrotubule agent includes at least one of vincristine, vinblastine and a taxane.

17. A method as set forth in claim 15, wherein the taxane includes at least one of paclitaxel and docetaxel.

18. A method as set forth in claim 12, wherein a therapeutic amount of the vitamin D conjugate provides an amount of the cytotoxic agent which is lower than the antiproliferative effective amount of the cytotoxic agent when administered alone.

19. A method as set forth in claim 1 wherein the cells are hepatic tumors and the target molecule includes a medium-chain triglyceride.

20. A method as set forth in claim 12, wherein the pathological effect is due to hepatic tumors and the target molecule includes a medium-chain triglyceride.

21. A compound of the formula
D-[(J)_{z}-L-AP]
wherein D is a vitamin D moiety; J is an acid cleavable linker which is covalently bonded to D; z is 0, 1 or 2; L is a linking moiety; and AP is a moiety which is a phosphonic acid ligand.

22. A compound as set forth in claim 21 wherein AP is selected from the group consisting of a 1-hydroxyethylidene-1-bisphosphonic ligand, a dichloromethylene bisphosphonic acid ligand, a 3-amino-1-hydroxypropylidene-1-bisphosphonic acid ligand and a polyaminomethylenephosphonic acid ligand.

23. A compound as set forth in claim 21, wherein the vitamin D moiety includes a moiety of at least one of 1α-dihydroxyvitamin D₂, 1α,2-dihydroxyvitamin D₄, 1α,24-dihydroxyvitamin D₂, 1α,25-dihydroxyvitamin D₃, la hydroxyvitamin D₃, 1α,25-dihydroxyvitamin D₂, 1α,25-dihydroxyvitamin D₄, 1α,24,25-dihydroxyvitamin D₂, seocalcitol, calcipotriol, maxacalcitol, falecalcitriol, and paricalcitol.

24. A method of site-specific delivery of a vitamin D moiety to bone in a patient, the method comprising administering to the patient a therapeutically effective dose of the compound of claim 21.

25. The method of claim 24, wherein AP is selected from the group consisting of a 1-hydroxyethylidene-1-bisphosphonic ligand, a dichloromethylene bisphosphonic acid ligand, a 3-amino-1-hydroxypropylidene-1-bisphosphonic acid ligand and a polyaminomethylenephosphonic acid ligand.

26. A pharmaceutical composition comprising the compound of claim 21 and a pharmaceutically acceptable vehicle.

27. A method of treating bone diseases in a human subject, comprising administering to the subject an effective amount of a vitamin D conjugate, the conjugate comprising a vitamin D moiety and a bone-seeking molecule moiety.

28. A method as set forth in claim 27, wherein the bone-seeking molecule is a peptide having an affinity for bone.

29. A method as set forth in claim 28, wherein the peptide is a small acidic peptide.

30. A method as set forth in claim 29, wherein the peptide is (Asp)₆ or (Glu)₆.

31. A method as set forth in claim 28, wherein the peptide is associated with osteonection, bone sialoprotein or osteopontin.

32. The method of claim 21, wherein the bone-seeking molecule includes at least one of tetracycline, DHEA, calcitonin, a bisphosphonate, a chelator, a phosphate, polyaspartic acid, polyglutamic acid, an aminophosphosugar, an estrogen, a peptide associated with mineral phase of bone such as osteonectin, bone sialoprotein and osteopontin, and a protein with bone mineral binding domains.

33. A method as set forth in claim 34, wherein the bisphosphonate includes at least one of alendronate, clodronate, etidronate, ibandronate, pamidronate, risedronate, tiludronate, zoledronate and combinations thereof.

34. A targeted delivery system comprising a vitamin D molecule bound to a targeting moiety.

35. A targeted delivery system as set forth in claim 34, wherein the vitamin D molecule includes at least one of 1α-dihydroxyvitamin D₂, 1α,2-dihydroxyvitamin D₄,1α,24-dihydroxyvitamin D₂, 1α,25-dihydroxyvitamin D₃, 1α hydroxyvitamin D₃, 1α,25-dihydroxyvitamin D₂, 1α,25-dihydroxyvitamin D₄, 1α,24,25-dihydroxyvitamin D₂, seocalcitol, calcipotriol, maxacalcitol, falecalcitriol, and paricalcitol.

36. A targeted delivery system as set forth in claim 34, wherein the targeting moiety includes DHEA.

37. A targeted delivery system as set forth in claim 34, wherein the targeting moiety includes a peptide.

38. A system as set forth in claim 37, wherein the peptide has an affinity to bone.

39. A system as set forth in claim 39, wherein the affinity is for hydroxyapatite (HA) formed in the osteoblasts of the matrix of bone.

40. A system as set forth in claim 39, wherein the peptide is a small acidic peptide.

41. A system as set forth in claim 40, wherein the small acidic peptide is (Asp)₆.

42. A system as set forth in claim 40, wherein the small acidic peptide is (Glu)₆.

43. A system as set forth in claim 37, wherein the peptide is a peptide associated with osteonectin, bone sialoprotein or osteopontin.

44. A system as set forth in claim 34, further comprising at least one therapeutic agent other than a vitamin D molecule.

45. A system as set forth in claim 44, wherein the therapeutic agent includes at least one of conjugated estrogens or their equivalents, antiestrogens, calcitonin, bisphosphonates, calcium supplements, cobalamin, pertussis toxin, boron, dehydroepiandrosterone, transforming bone growth factor beta, activin, and bone morphogenic protein.

46. A system as set forth in claim 44, wherein the therapeutic agent includes at least one of estromustene phosphate, prednimustine, cisplatin, S-fluorouracil, melphalan, hydroxyurea, mitomycin, idarubicin, methotrexate, adriamycin and daunomycin.

47. A pharmaceutical composition comprising:
a conjugate which includes at least one vitamin D moiety associated with at least one peptide having an affinity for bone, and a pharmaceutically acceptable carrier.

48. The pharmaceutical composition of claim 47, where in affinity of the peptide is for the matrix of bone wherein hydroxyapatite (HA) is formed in the osteoblasts.

49. The pharmaceutical composition of claim 47, wherein the peptide includes a small acidic peptide.

50. The pharmaceutical composition of claim 49, wherein the small acidic peptide includes (Asp)₆.

51. The pharmaceutical composition of claim 49, wherein the small acidic peptide includes (GIu)₆.

52. The pharmaceutical composition of claim 47, wherein the peptide includes a peptide associated with osteonectin, bone sialoprotein or osteopontin.

53. The pharmaceutical composition of claim 47, further comprising a differentially degradable coating encapsulating the conjugate for time release delivery of the conjugate.

54. A kit for the targeted delivery of an active vitamin D molecule, comprising:
at least one dosage unit containing a composition comprising a conjugate of a targeting moiety and a vitamin D moiety.

55. The kit of claim 54, wherein the targeting moiety includes a bone seeking agent.

56. A method of treating tumors, comprising: administering to a patient a composition comprising a conjugate of an antibody, or a fragment or a derivative thereof, and a vitamin D molecule in a therapeutically effective dosage.

57. A method of targeted delivery of a therapeutic agent to cancer tissue comprising:
conjugating an active vitamin D compound to an antibody which binds to a cell-surface antigen of cells of the cancer tissue and administering the conjugated antibody to a patient.
